(19) United States
(12) Patent Application Publication
(10) Pub. No.: US 2003/0017169 A1
(43) Pub. Date: Jan. 23, 2003

(54) Controlled release systems for polymers

(76) Inventor: Sidney Pestka, North Caldwell, NJ (US)

Correspondence Address: ROPES & GRAY, ONE INTERNATIONAL PLACE, BOSTON, MA, 02110-2624, US

(21) Appl. No.: 10/193,654

(22) Filed: Jul. 11, 2002

Related U.S. Application Data
(63) Continuation-in-part of application No. 10/040,267, filed on Dec. 31, 2001.
(60) Provisional application No. 60/258,916, filed on Dec. 29, 2000.

(51) Int. Cl. A61K 39/00

(52) U.S. Cl. 424/185.1

(57) ABSTRACT

The present invention relates to controlled release delivery of biologically active molecules from a solid composition prepared by exposure of the molecules to an organic compound. For instance, the organic compound is an organic solvent, such as an alcohol (e.g., preferably a lower alcohol, such as methanol, ethanol, isopropanol, n-propanol, n-butanol, isobutanol, t-butanol, etc.), a mixture of alcohols, an aldehyde, a ketone, a hydrocarbon (saturated or unsaturated), or an aromatic hydrocarbon. The solvent can be a mixture of different organic solvents, or the resulting formulation can be a mixture of, e.g., different lyophilized preparations, such as may be used to control the release profile of the resulting admixture.

### REFERENCE TO RELATED APPLICATIONS

This application is a continuation-in-part of, and claims priority to, U.S. patent application Ser. No. 10/040267, filed Dec. 31, 2001, the specification of which is incorporated by reference herein, and which claims priority to U.S. Provisional Patent Application No. 60/258,916 filed on Dec. 29, 2000, the specification of which is also incorporated by reference herein.

### BACKGROUND OF THE INVENTION

With the advent of genetic engineering, the large-scale availability of many bioactive polymers, such as proteins, carbohydrates and nucleic acids, has been achieved. However, the administration of these recombinantly produced peptides and proteins presents a unique set of problems. In many cases the maintenance of the biological effect of these proteins requires long-term administration. Daily administration of these agents in aqueous vehicles is inconvenient and costly; sustained or prolonged release is preferred. In addition, proteins are highly unstable in an aqueous environment most suitable for administration.

Moreover, successful treatment of a variety of conditions is limited by the fact that agents known to effectively treat these conditions may have severe side effects, requiring low dosages to minimize these side effects. In other instances, the therapeutic agents may be very labile, or have very short half-lives requiring repeated administration. In still other instances, the long term administration of a pharmaceutical agent may be desired.

In all these cases, the ability to deliver a controlled dosage in a sustained fashion over a period of time may provide a solution.

### SUMMARY OF THE INVENTION

One aspect of the present invention relates to controlled release delivery of biologically active molecules from a solid composition prepared by exposure of the molecules to an organic compound. For instance, the organic compound is an organic solvent, such as an alcohol (e.g., preferably a lower alcohol, such as methanol, ethanol, isopropanol, n-propanol, n-butanol, isobutanol, t-butanol, etc.), a mixture of alcohols, an aldehyde, a ketone, a hydrocarbon (saturated or unsaturated), or an aromatic hydrocarbon. The solvent can be a mixture of different organic solvents, or the resulting formulation can be a mixture of, e.g., different lyophilized preparations, such as may be used to control the release profile of the resulting admixture.

The subject molecule to be formulated for controlled release can be an organic compound. In certain embodiments, it is a polymer, preferably a biopolymer such as a protein, a peptide, a nucleic acid, an oligonucelotide, a carbohydrate, a ganglioside, or a glycan. The subject molecule can be a lipid, a sterol or other lipophilic moiety. The subject controlled delivery system can be used to deliver the controlled release of small molecules (e.g., organic compounds).

In certain embodiments, the subject preparations are prepared by precipitation and/or lyophilization.

### BRIEF DESCRIPTION OF THE DRAWINGS

FIGS. 1-5. Graphs showing various release profiles for BSA preparations.

FIGS. 6A-D. Effect of salt concentration of formulation on release of HSA and IFN-α012. Solution I consisted of 9.0 mg of HSA (Immuno-U.S.) and 10 μg of IFN-α012 in 40% (w/w) n-propanol (0.364 g n-propanol) in H₂O for a total weight of 0.91 g. The various Solution II compositions consisted of various quantities of sodium acetate (1 M, pH 6.3) and deionized water and 0.040 g n-propanol to make solutions of 40% n-propanol and 250, 450, and 600 mM final sodium acetate concentrations with a total volume of 0.10 g. Solution II (0.10 g) was added to Solution I (0.91 g) with stirring to yield a final 1.01 g of each formulation. The final 1.01 g formulations containing 40% n-propanol and 25, 45, and 60 mM concentrations of sodium acetate were stirred in 2 ml glass vials for 6 hours at 24° C. and passed through 25G syringe needles just prior to separating supernatants from precipitates. The quantity of HSA and IFN-α012 in washed precipitates was determined as described in Materials and Methods. Release was performed in PBS/0.01% thimerosal. A & B. Absolute (mg) and percent release of precipitated HSA, respectively. C & D. Absolute (ng) and percent release of precipitated IFN-α012, respectively.

FIGS. 7A-B. Effect of cation species in formulation on release of HSA. Solution I consisted of 8.1 mg of HSA (Immuno-U.S.) in 40% (w/w) n-propanol in deionized water in a total volume of 0.91 ml. The various Solution II compositions consisted of adding none or 0.025 ml of various salt stocks (each at 1 M cation concentration, pH 6.3) to deionized water followed by n-propanol to make solutions 40% (w/w) n-propanol and 250 mM final cation concentration in a total volume of 0.10 ml. Solution II (0.10 ml) was added to 0.91 ml of Solution I with stirring to give a final 1.01 ml formulation having 40% (w/w) n-propanol. The final 1.01 ml formulations containing 40% n-propanol and no or 25 mM concentrations of potassium, sodium or magnesium acetate were stirred in 2 ml glass vials for 6 hours at 24° C. prior to separating supernatants from precipitates. The quantity of HSA in washed precipitates was determined as described in Materials and Methods. Release was performed in PBS/0.01% thimerosal. A & B. Absolute (mg) and percent release of precipitated HSA, respectively. Salts were sodium, potassium, and magnesium acetate (indicated by NaOAc, KOAc, and Mg(OAc)₂, respectively).

FIG. 8A-B. Effect of cation species in formulation on release of IFN-α012. Solution I consisted of 45 mg of HSA (Immuno-U.S.) and 5.44 μg IFN-α012 in 40% (w/w) n-propanol in deionized water in a total volume of 4.55 ml. The various Solution II compositions consisted of adding 36 μl of 0.1 M acetic acid (to compensate for the buffer capacity of the HSA solution) and 0.250 g of potassium, sodium or magnesium acetate solution (each at pH 6.3) to 0.314 g of deionized water and 0.400 g of n-propanol to make solutions of 40% (w/w) n-propanol and 250 mM final acetate concentration in a total weight of 1 g. The potassium acetate solution was made with 0.980 g potassium acetate, 10.061 g water and 0.274 ml 1 M acetic acid. The sodium acetate solution was made with 0.823 g sodium acetate, 10.056 g water and 0.245 ml 1 M acetic acid. The magnesium acetate solution was made with 2.144 g magnesium acetate, 10 g water and 0.200 ml 1 M acetic acid. Solution II (0.50 ml) was added to 4.55 ml of Solution I with stirring to give a final 5.05 ml formulation having 40% (w/w) n-propanol. The final formulations were stirred in 50 ml conical tubes for 6 hours at 24° C., the precipitates washed with 5 ml of PBS/0.01% thimerosal, then suspended in 5 ml PBS/0.01% thimerosal, then split into two individual 2.5 ml samples prior to separating supernatants from precipitates. Release data is from the precipitates from one 2.5 ml portion of the formulation. The amount of IFN-α012 in washed precipitates was determined as described in Materials and Methods. Release was performed in PBS/0.01% thimerosal. A & B. Absolute (ng) and percent release of precipitated IFN-α012, respectively. Salts were sodium, potassium, and magnesium acetate (indicated by 21 mM NaOAc, 20 mM KOAc, and 18 mM Mg(OAc)₂, respectively).

FIGS. 9A-B. Effect of aqueous solution pH of formulation on release of IFN-α012. Acetic acid (0.1 M) was used to adjust 5% HSA (Alpha Therapeutic) stock solutions to pH 5.0 or pH 7.0. Solution I consisted of 10 mg of HSA from either pH 5.0 or pH 7.0 HSA stock solutions, 6.83 μg IFN-α012 and additional water to a total weight of 0.6 g. The final formulations were prepared by adding 0.4 g of n-propanol to Solution I with stirring to yield a concentration of 40% (w/w) n-propanol. Final 1 g formulations were stirred in 2 ml glass vials for 24 hours at 24° C. prior to separating supernatants from precipitates. The quantity of IFN-α012 in washed precipitates was determined as described in Materials and Methods. Release was performed in PBS/0.01% thimerosal. A & B. Absolute (ng) and percent release of precipitated IFN-α012, respectively.

FIG. 10A-B. Effect of aqueous solution pH of formulation on release of HSA and IFN-α012. Solution I consisted of 45 mg of HSA (Immuno-U.S.) and 5.44 μg IFN-α012 in 40% (w/w) n-propanol in deionized water in a total volume of 4.55 ml. Solution II compositions were prepared as follows. Solution IIa: 1.55 ml of 1 M acetic acid was added to 0.82 g anhydrous sodium acetate and 10 g deionized water to adjust pH of this Solution A to 5.52; then 0.036 ml of 0.1 M acetic acid was added to 0.250 g of Solution A to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. Solution IIb: 0.40 ml of 1 M acetic acid was added to 0.82 g anhydrous sodium acetate and 10 g of deionized water to adjust pH of this Solution B to 6.13; then 0.036 ml of 0.1 M acetic acid was added to 0.250 g of Solution B to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. Solution IIc: 0.245 ml of 1 M acetic acid was added to 0.823 g anhydrous sodium acetate and 10.056 g deionized water to adjust pH of this Solution C to 6.31; then 0.036 ml of 0.1 M acetic acid was added to 0.250 g of Solution C to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. To prepare the final formulations, 0.50 ml from Solutions Ia, IIb, or IIc was added to 4.55 ml of Solution I with stirring to yield three 5.05 ml formulations having 40% (w/w) n-propanol and pH 5.52, pH 6.13 or pH 6.31, respectively. Final formulations were stirred in 50 ml conical tubes for 6 hours at 24° C., then split into two individual 2.52 ml samples prior to separating supernatants from precipitates. Release data is from one 2.52 ml portion of the formulation. The amount of IFN-α012 in washed precipitates was determined as described in Materials and Methods. Release was performed in PBS/0.01% thimerosal. A & B. Absolute (mg) and percent release of precipitated HSA, respectively. C & D. Absolute and percent release of precipitated IFN-α012, respectively.

FIG. 11A-B. Effect of acid concentration of formulation on release of HSA and IFN-α001 from precipitates formed in the presence of 25 mM sodium acetate. Solution I consisted of 8.1 mg of HSA (Immuno-U.S.) and 0.92 μg IFN-α001 in 40% (w/w) n-propanol in deionized water in a total volume of 0.9 ml. Several Solution II formulations, IIa, IIb, IIc and IId, were prepared consisting of 0.004, 0.010, 0.015 and 0.025 ml of 0.1 M acetic acid, respectively, in 40% (w/w) n-propanol in deionized water. Solution III consisted of 1 M sodium acetate and 40% (w/w) n-propanol in deionized water in a total volume of 0.025 ml. Several Solution IV formulations, IVa, IVb, IVc and IVd, were prepared consisting of 0.071, 0.065, 0.060 and 0.050 ml of 40% (w/w) n-propanol, respectively, in deionized water. In preparing the final formulations, Solutions Ia, IIb, IIc and IId were matched with Solutions IVa, IVb, IVc and IVd, respectively. Solutions II, III and IV were mixed together then Solution I added rapidly to the mixture to give a final 1 ml formulation. This yielded a formulation having a final concentration of 25 mM sodium acetate, 40% (w/w) n-propanol and the final acetic acid concentrations indicated on the Figure. Formulations were stirred in 2 ml glass vials for 6 hours at 24° C. prior to separating supernatants from precipitates. After washing, precipitates were lyophilized 4 hours at <400 mTorr. The amount of HSA in washed precipitates was determined as described in Materials and Methods. Release was performed in PBS/0.01% thimerosal. A & B. Absolute (mg) and percent release of precipitated HSA, respectively. C & D. Absolute (ng) and percent release of precipitated IFN-α012, respectively.

FIG. 12A-D. Effect of salt concentration of formulation on release of HSA and IFN-α001 from precipitates formed in the presence of 1.5 mM acetic acid. Solution I consisted of 8.1 mg of HSA (Immuno-U.S.) and 0.92 μg IFN-α001 in 40% (w/w) n-propanol in deionized water in a total volume of 0.9 ml. Solution II consisted of 0.1 M acetic acid and 40% (w/w) n-propanol in deionized water in a total volume of 0.015 ml. Several Solution III formulations, IIIa, IIIb, IIIc and IIId, were prepared consisting of 0, 0.015, 0.025 and 0.035 ml of 1 M sodium acetate, respectively, in 40% (w/w) n-propanol in deionized water. Several Solution IV formulations, IVa, IVb, IVc and IVd, were prepared consisting of 0.085, 0.070, 0.060 and 0.050 ml of 40% (w/w) n-propanol, respectively, in deionized water. In preparing the final formulations, Solutions IIIa, IIIb, IIIc and IIId were matched with Solutions IVa, IVb, IVc and IVd, respectively. Solutions II, III and IV were mixed together then Solution I added rapidly to the mixture to give a final 1 ml formulation. This yielded a final concentration of 1.5 mM acetic acid, 40% (w/w) n-propanol (w/w) and the final sodium concentrations indicated on the Figure. Formulations were stirred in 2 ml glass vials for 6 hours at 24° C. prior to separating supernatants from precipitates. After washing, precipitates were lyophilized 4 hours at <400 mTorr. The amounts of HSA and IFN-α001 in washed precipitates were determined as described in Materials and Methods. Release was performed in PBS/0.01% thimerosal. A & B. Absolute (mg) and percent release of precipitated HSA, respectively. C & D. Absolute (ng) and percent release of precipitated IFN-α001, respectively.

FIG. 13A-B. Effect of salt concentration and pH of formulation on release of HSA with tertiary butanol precipitates. Acetic acid (0.1 M) was used to adjust 5% HSA stock solutions (Alpha Therapeutic) to pH 5.35 or 7.0. Solution I consisted of 18.0 mg of HSA from the pH 5.35 or pH 7.0 5% stock solution, 1.0 μg IFN-α012 and deionized water bringing the total solution weight to 0.375 g. To prepare Solutions Ia and IIb with NaCl concentrations of 0.02 M and 0.1 M, respectively, sufficient deionized water was added to 0.021 and 0.0043 ml of a 3.75 M NaCl solution to bring the total weight of each solution to 0.425 g. Both pH 5.35 and pH 7.0 variants of Solution I (0.375 g) were added to Solutions Ia and IIb to yield 0.80 g of the various combinations of pH and NaCl concentration as shown in the Figure prior to the addition of 0.31 or 0.47 g of tert-butyl alcohol to yield 28.1% and 36.9% (w/w) tert-butyl alcohol (see summary of the chart legends). Final 1.11-1.27 g formulations were stirred in 2 ml glass vials for 24 hours at 24° C. prior to separating supernatants from precipitates. The amount of HSA in washed precipitates was determined as described in Materials and Methods. Release was performed in PBS/0.01% thimerosal. A & B. Absolute (mg) and percent release of precipitated HSA, respectively.

FIG. 14. Effect of pH and salt concentration of formulation on threshold of precipitation of HSA by n-propanol. An 11% (w/w) HSA (USB) was dialyzed 3 times for 6 hours each time against 2 L deionized H₂O in a Pierce Slide_alyzer (15 ml capacity, No. 66410, lot # BJ44820B). The final concentration was analyzed by spectrophotometry at 280 nm to be 8.28% (w/w). This solution was diluted to 4% (w/w) with deionized water. Amounts (0.9 g) of 4% HSA were weighed into 2 ml glass vials. Sodium acetate (1 M), acetic acid (1 M), sodium hydroxide (1 M), and water were added in various combinations in a total weight of 0.1 g to yield the final sodium concentrations and pH values, measured in 1 g formulations as shown in the Figure. Subsequently, n-propanol was added in about 50 μl increments with stirring, and the point at which initial precipitates were stable (did not re-dissolve with stirring within 5 minutes) was recorded. Connected data points indicate equivalent sodium concentrations at various pH and n-propanol (w/w) concentrations.

FIG. 15. Effect of n-propanol concentration on the formation of human IFN-α001 particles at various pH and buffers. The final concentration of human IFN-α001 was 0.156 mg/ml with 2.2 mM Tris HCl, 24 mM NaCl, 0.5% glycerol, in either 25 mM NaOAc pH 5.2 (solid squares), 25 mM NaOAc pH 6.2 (solid triangles), 25 mM NaOAc pH 7.6 (solid circles), 25 mM NaPO₄ (open triangles), 25 mM Tris HCl (open circle). Formation of human IFN-α001 particles was monitored by increase of absorbance at 405 nm using a microplate aggregation assay.

FIG. 16. Effect of different wash buffers and release conditions on protein release from a single set of formulation conditions. Percent of the protein released from the pellet was determined for HSA/α001 formulation washed with PBS, released in PBS Run 1 (open circles), Run 2 (solid circles); HSA/α001 PBS washed and released in 3% Media Run 1 (open triangles), Run 2 (solid triangles); HSA/α012 formulation washed with NaOAc, released in PBS Run 1 (open squares), and HSA/α001 formulation washed with NaOAc, released in PBS Run 2 (solid squares).

FIG. 17A-B. Release of total and immunoreactive protein from human IFN/HSA formulations. A. Percent release of protein for human IFN-α001 (solid circles) and human IFN-α012 (solid triangles) was determined by BioRad Assay. Percent release of immunoreactive material for human IFN-α001 (open circles) and human IFN-α012 (open triangles) was determined by ELISA. B. Relationship of ELISA-reactive Interferon released to total protein released from IFN-α001/HSA (light bars) and IFN-α012/HSA (dark bars) formulations. The amount of ELISA-reactive material released at each time point was divided by the total protein released as determined by BioRad Microassay.

FIG. 18. Comparison of release of small molecule dyes from HSA coformulations. Interval (medium gray) and cumulative (dark gray) release of Cibacron Blue from HSA coformulations is compared with interval (light gray) and cumulative (black) release of Green 5 from HSA coformulations.

FIG. 19. Formation of human IFN-α001 particles in the presence of potential stabilizers. Particle formation was monitored by measuring absorbance at 405 nm in the microplate assay. All samples contained 0.225 mg/ml human IFN-α001, 25 mM NaOAc pH 5.4, 25 mM NaCl, 0.2% glycerol final concentration. The final formulations were prepared by mixing 100 μl of a 2× concentrated stock of the interferon in buffer and any stabilizers with 100 μl of a 2× concentrated propanol/water solution to initiate particle formation. The final conditions tested were: 25 mM NaCl (solid circles), 50 mM NaCl (open circles), 100 mM NaCl (gray circles), 10 mM MgSO₄ (solid squares), 0.5% Dextran 500,000 (open triangles), 1.8% Trehalose (solid triangles), 0.5% Tween 80 (solid diamonds), and 0.05% SDS (open diamonds).

FIG. 20. Theoretical combination of two formulations to alter pattern of protein release. For Formulation I (filled squares), Solution I consisted of 5 mg HSA (100 μl of 5% (w/v) HSA stock) and 47 μg of IFN-α001 (50 μl of 0.94 mg/ml stock). To this was added with stirring 59 μl H₂O, 90 μl of 0.01 M acetic acid, 16 μl 1M NaCl, and finally 237 μl t-butanol. The final formulation was stirred in a 2 ml glass vial for 22 hours at 24° C. prior to separating supernatant from precipitate. For Formulation II (filled circles), Solution I consisted of 5 mg HSA (0.100 μl of 5% HSA stock) and 47 μg of IFN-α001 (50 μl of 0.94 mg/ml stock). To this was added with stirring 59 μl H₂O, 16 μl 1M NaCl, and finally 237 μl t-butanol. The final formulation was stirred in a 2 ml glass vial for 6 hours at 24° C. prior to separating supernatant from precipitate. The quantity of protein in washed precipitates was determined as described in Materials and Methods. Release was performed in PBS/0.01% thimerosal. Theoretical release (open triangles) that would be obtained by mathematically averaging these two formulations is also presented. A & B. Absolute (mg) and percent release of precipitated protein, respectively.

FIG. 21. Release of a peptide and two small molecules from HSA co-formulations.

Release of Cibacron Blue (open circles), Green 5 (solid triangles) and a peptide Caspase I substrate (open squares) from HSA co-formulations was monitored by absorbance of the Cibacron Blue and Green 5 at 650 nm and the peptide Caspase 1 substrate at 494 nm.

### DESCRIPTION OF THE INVENTION

I. Overview

The present invention relates to a controlled release delivery system and is based on the discovery that treatment of proteins and other molecules such as carbohydrates, nucleic acids, and other substances with organic compounds can modify their solubility in aqueous media. For example, in one embodiment the exposure of the proteins to the organic solvent (such as an alcohol) replaces the water molecules and other associated moieties with organic residues. In certain embodiments, the subject preparations are solids, e.g., powders or crystals formed by lyophilization, precipitation or the like.

The resulting preparations can provide prolonged release formulations of the proteins, e.g., suitable for sustained biological effects when used as pharmaceuticals or in other aqueous uses. The examples given refer to protein, but the principle can apply to other water soluble biopolymers as well such as peptides, carbohydrates, nucleic acids, oligonucleotides, lipids, glycans, gangliosides and other biopolymers. Small organic molecules and some inorganic molecules that are solvated with attached water residues can be treated in an analogous way to provide controlled delivery of the specific molecules.

In certain embodiments, a controlled release system may comprise a first biopolymer and a therapeutic agent such as a second biopolymer, a small organic molecule or a small inorganic molecule. Optionally, the first biopolymer acts as a carrier for the therapeutic agent, thus permitting the formation of a controlled release system regardless of whether or not the therapeutic agent is, on its own, suitable for formation of a controlled release system of the invention. Optionally, the first biopolymer also provides a therapeutic effect.

Furthermore, solubility of proteins is also modulated by porttranslational modifications that can change the solubility of the proteins. The methods described can alter the solubility of the proteins with and without the post-translational modifications.

In certain embodiments, the biomolecules are precipitated from the aqueous solution by addition of organic solvents and then lyophilized. In alternative procedures, the solution can be lyophilized directly from solution containing organic solvents to provide for the dried material to be formulated into a controlled release system; the precipitated protein washed with aqueous solution and then formulated directly without lyophilization; or the dry protein treated with organic solvent, then formulated after removal of the solvent.

In certain preferred embodiments, the solvent is a an inert solvent, and even more preferably an anhydrous organic solvent. The solvent should not irreversibly denature the polymer, e.g., the timescale for renaturation, if any is requireed, should not be signiificantly longer than the rehydration process.

Formulation and size of the material can be controlled by the timing and method of precipitation and lyophilization conditions. Upon precipitation of the molecules, the precipitate is lyophilized to remove excess water and prevent water from immediately replacing the organic solvents. Colloidal suspensions without direct precipitation can be used to substitute for precipitation. The colloidal suspensions can be used to generate particles of small size. Furthermore, the mixtures can be lyophilized directly without precipitation or colloid formation to provide particles of different sizes dependent on the concentration of the molecules in the organic-aqueous media, the method of precipitation and the concentration of the protein solution. In some instances, inorganic molecules that can replace the water molecules on the molecules to be released slowly can be used in a total aqueous system to provide the same results. after lyophilization. The release is affected by the-specific organic solvent used, the buffer used, and the particle size of the precipitated and/or lyophilized protein.

In addition, the method of invention permits greater tailoring of release profiles. The subject preparations can be made to exhibit short-term or long-term release kinetics, thereby providing either rapid or sustained release of macromolecules. In any event, the subject preparations have, relative to preparations of the polymer lyophilized from aqueous solutions, a reduced solubility in serum or other biological fluid, e.g., the solubility rate over a period of at least 24, 48, or even 168 hours (7 days) is at least 2 fold less than preparations of the polymer lyophilized from aqueous solution, and more preferably at least 10, 25, 50 or even 100 fold less. In certain embodiments, a formulation having desired release profiles may be generated by mixing formulations prepared by different methods and having different release profiles. For example, a formulation having, on average, a quicker release may be mixed with a formulation having, on average, a slower release to provide a combination formulation having a more consistent overall release profile.

In certain preferred embodiments, the subject compositions permit the release of biologically active compound at a rate which provides an average steady state dosage of at least the ED₅₀ for the active compound for a period of at least 2 days, and more preferably at least 7, 14, 21, 50, or even 100 days.

In certain preferred embodiments, the solvent(s) are chosen such that, when administered to a patient (particularly a human), the solvent released from the formulation is done so at a rate which remains below the IC₅₀ for deleterious side effects, if any, of the solvent, and more preferably at least 1, 2 or even 3 orders of magnitude below such IC₅₀ concentrations.

In certain embodiments, the organic agent is a polar protic solvent, such as for example, aliphatic alcohols, glycols, glycol ethers, and mixtures thereof. In certain preferred embodiments, the organic agent is a water-miscible polar protic solvent.

Biodegradable or non-biodegradable materials known in the art in the form of gels, microspheres, wafers or inplants can be mixed with the subject modified molecules.

These subject formulations can be used in parenteral, oral, intramuscular, subcutaneous, dermal, intravenous, intrarterial, intralesional, intrathecal or other sites of delivery for the treatment, prevention and diagnosis of many diseases.

Still another aspect of the invention relates to a method for doing business, e.g., for the preparation of pharmaceutical formulations for the treatment of humans or other animals. In an exemplary embodiment of such methods, there is provided a lyophilization facility for generating the lyophilized preparations described herein. The lyophilized preparations are packaged as e.g., pills, tablets, patches, injectables and the like, preferably at a government approved facility, e.g., an FDA-approved facility. In preferred embodiments, the lyophilized preparation is provided in single dosage form, even if packaged in larger lots.

II. Definitions

“Bioerodible” signifies that the material may be dissolved or digested into component molecules by the action of the environment or particularly by the action by living organisms, and optionally metabolized or digested into simpler constituents without poisoning or distressing the environment or the organism.

“Administered to a mammal” means that the composition containing an active ingredient is administered orally, parenterally, enterically, gastrically, topically, transdermally, subcutaneously, locally or systemically. The composition may optionally be administered together with a suitable pharmaceutical excipient, which may be a saline solution, ethyl cellulose, acetotephtalates, mannitol, lactose, starch, magnesium stearate, sodium saccharin, talcum, glucose, sucrose, carbonate, and the like.

“Sustained delivery” or “sustained time release” denotes that the active ingredient is released from the delivery vehicle at an ascertainable and manipulatable rate over a period of minutes, hours, days, weeks or months, ranging from about thirty minutes to about two months or longer.

| Abbreviations | |
|---|---|
| HSA | Human serum albumin |
| HOAc | Acetic acid |
| NaCl | Sodium chloride |
| NaOAc | Sodium acetate |
| KOAc | Potassium acetate |
| Mg(OAc)₂ | Magnesium acetate |
| IFN-α001 | Interferon α-001 |
| IFN-α012 | Interferon α-012 |
| PBS | Phosphate-buffered saline |

III. Exemplary Biopolymers

The biopolymers which may be used in the present invention include proteins, carbohydrates, nucleic acids and combinations thereof.

Advantageously, according to the present invention, the subject method can be used to formulate a protein which is pharmaceutically valuable or of value in the agri-foodstuffs industry. Proteins of interest include cytokines, growth factors, somatotropin, growth hormones, colony stimulating factors, , erythropoietin, plasminogen activators, enzymes, T-cell receptors, surface membrane proteins, lipoproteins, clotting factors, anticlotting factors, tumor necrosis factors, transport proteins, homing receptors, addressins, etc. Examples of mammalian polypeptides include molecules such as renin, a growth hormone, including human growth hormone; bovine growth hormone; growth hormone releasing factor; parathyroid hormone; thyroid stimulating hormone; lipoproteins; α-1-antitrypsin; insulin; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-α and -β; enkephalinase; RANTES (regulated on activation normally T-cell expressed and secreted); human macrophage inflammatory protein (MIP-1-α); a serum albumin such as human serum albumin; mullerian-inhibiting substance; relaxin A-chain; relaxin B-chain; prorelaxin; mouse gonadotropin-associated peptide; a microbial protein, such as beta-lactamase; DNase; inhibin; activin; vascular endothelial growth factor (VEGF); receptors for hormones or growth factors; integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factors (TGF) such as TGF-α, TGF-β and BMPs; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; antigens (e.g., bacterial and viral antigens); transport proteins; homing receptors; addressing; regulatory proteins; immunoglobulin-like proteins; antibodies; nucleases; and fragments of any of the above-listed polypeptides.

Other examples of suitable therapeutic and/or prophylactic biologically active agents include nucleic acids, such as antisense molecules; and small molecules, such as antibiotics, steroids, decongestants, neuroactive agents, anesthetics, sedatives, cardiovascular agents, anti-tumor agents, antineoplastics, antihistamines, hormones (e.g., thyroxine) and vitamins.

IV. Exemplary Methods

The rate of controlled release of the protein can be modified by many variables. The variables include rate of addition of organic solvent, time of protein (or other molecule) in organic solvent (time of exposure of protein to organic solvent), concentration of organic solvents for precipitation of the protein, concentration of the organic solvents prior to precipitation, concentration of the organic solvents prior to lyophilization from solution directly, organic and non-organic composition of media, temperature, concentration of cations, concentration of anions, rate of precipitation, pH, mixtures of organic solvents, stirring, agitation, presence of other proteins as carriers, presence of other proteins for controlled release of multiple proteins, protein stabilizers, dissolved gasses, reducing agents, oxidizing agents, mass to surface area of the particles, washing of samples prior to preparation for release, salt concentration, length of time exposed to modifier agents, concentration of the proteins or other polymer, inorganic compounds, type of organic compounds, for example. Inorganic cations can be monovalent, divalent, trivalent, tetravalent or pentavalent; inorganic anions can be monovalent, divalent, trivalent, tetravalent or pentavalent. Optionally the aqueous solution comprises one or more of the following components: a salt, an organic acid and a stabilizer that may assist in preventing loss of activity of a biologically active compound. Exemplary salts include, but are not limited to, salts having a cationic portion selected from the group consisting of: lithium, sodium, potassium, magnesium, calcium, barium and ammonium, and an organic cation, (e.g. Tris, lysine, etc.) and an anionic portion selected from the group consisting of: carbonate, nitrate, fluoride, chloride, sulfate, phosphate and acetate (or another organic anion). In certain embodiments, sodium acetate or sodium chloride is the preferred salt. A mixture of any of the above salts may also be employed. Salt concentrations may be selected as appropriate for the biomolecule. Optionally, salt concentrations may range from about 5 mM to about 100 mM, optionally from about 20 mM to about 60 mM, and, in certain embodiments, the salt concentration is preferably about 25 mM. Exemlary organic acids include carboxylic acids such as acetic acid and formic acid. Optionally, the organic acid concentration may range from about 0.1 to 10 mM, and preferably about 2.5 mM. It is understood that, in an aqueous solution, organic acids and salts dissociate and reassociate with various counterions and other solution components, and therefore the description of the concentrations of various solution components herein should be understood to refer to the calculated concentrations based on the amounts of the components mixed into the solution. Exemplary stabilizers are described below. In certain embodiments, the pH of an aqueous solution, prior to addition of an organic solvent, is between about 4 and about 9, optionally between about 5 and about 7, and in certain preferred embodiments, the pH is between about 5 and about 6.

In some embodiments, lyophilization can be omitted. For example, the precipitate can be washed with a nonpolar solvent such as n-hexane to remove the organic solvent without affecting the protein; or the precipatate can be washed with an aqueous medium to remove the organic solvent removing the excess organic solvent from the protein mass. Furthermore, the precipitate can be washed and/or preincubated to remove soluble protein and eliminate the higher initial release rate.

Organic compound does not need to be solvent, just constituent in the mixture.

In addition, the protein precipitates can be placed into a variety of biodegradable or non-biodegradable materials known in the art in the form of gels, microspheres, wafers or implants. In these cases, the release is controlled by both the intrinsic protein release rate and the rate of release controlled by the gels, microspheres, wafers or implants. These formulations can be used in parenteral, oral, intramuscular, subcutaneous, dermal, intravenous, intrarterial, intralesional, intrathecal or other sites of delivery for the treatment, prevention and diagnosis of many diseases.

During equilibration of the protein with the solvent, the organic solvent used is attached to the protein in the precipitates. The organic solvent can be replaced partially or completely with other organic compounds soluble in the solution. The organic compounds can be active pharmaceuticals such as antibiotics, antimicrobial agents, aminoglycosides, chloramphenicol, macrolides, antifungals, cephalosporins, 3,4-dihydroxyphenylalanine (DOPA), adrenergic agonists, adrenergic antagonists, cholinergic agonists, cholinergic antagonists, muscarinic agonists, muscarinic antagonists, antiviral agents, sympathomimetics, sympatholytics, serotonin agonists, serotonin antagonists, antihypertensive agents, monoamine oxidase inhibitors, diuretics, antiarrhythmic drugs, phosphodiesterase inhibitors, digitalis glycosides, calcium antagonists, vasodilators, prostaglandins, autacoids, lipid lowering drugs, anticoagulants, fibrinolytics, platelet aggregation inhibitors, antidepressants, benzodiazepines, antiepileptics, antiparkinson agents, analgesics, opioids, opioid peptides, opiates, peptides, antiinflammatory drugs (NSAIDs, acetaminophen), barbiturates, peptide hormones, steroids, glucocorticoids, mineralocorticoids, estrogens, progestins, androgens, antiandrogens, thyroxine, triiodothyronine, cyclooxygenase inhibitors, growth hormone releasing hormone (GHRH), antineoplastic drugs, and antihistamines. The attached organic compounds (as drugs) linked to bovine or human serum albumin or other proteins such as immunoglobulins can then be delivered as the protein is released and dissolved. The proteins with attached organic solvents are thus able to be used as effective delivery systems. Furthermore, with the use of immunoglobulins and other proteins that can target to specific tissues or cells, the attached molecules can then be delivered to the tissues or cells.

Preparations made by the subject process can be either homogeneous or heterogeneous mixtures of active agents, or of preparations of active agents prepared under different conditions (e.g., using different solvents, etc).

The amount of a biologically active agent, which is contained in a specific preparation, is a therapeutically, prophylactically or diagnostically effective amount, which can be determined by a person of ordinary skill in the art taking into consideration factors such as body weight, condition to be treated, type of polymer used, and release rate from the preparation.

The biologically active agent can also be mixed with other excipients, such as stabilizers, surfactants, solubility agents. Stabiliz a added to-maintain the potency of the agent over the duration of the agent's release. Suitable stabilizers include, for example, carbohydrates, amino acids, fatty acids and surfactants and are known to those skilled in the art. Solubility agents are added to modify the solubility of the agent in aqueous solution or, as the case may be, in organic solvents. Suitable solubility agents include complexing agents, such as albumin and protamine, which can be used to control the release rate of the agent. Bulking agents typically comprise inert materials. Further exemplary stabilizers include dextran (e.g. dextran with an average molecular weight of 500,000 D), trehalose, Tween 80 and sodium dodecyl sulfate.

In another embodiment, a biologically active agent can be lyophilized with a metal cation component, to further stabilize the agent and control the release rate of the biologically active agent.

In certain embodiments, precipitates, lyophilates and crystals produced according to the methods described herein may be washed prior to use. Washing may be done with an aqueous solution or an organic solution. Exemplary aqueous wash solutions include PBS and a solution comprising NaOAc.

The subject formulations, if used a therapeutics, may be administered to a human or animal by oral or parenteral administration, including intravenous, subcutaneous or intramuscular injection; administration by inhalation; intraarticular administration; mucosal administration; ophthalmic administration; and topical administration. Intravenous administration includes catheterization or angioplasty.

In other embodiments, the subject preparations can be used in non-therapeutic aqueous environments, such as for the release of agents (such as enzymes) into a water supply or water treatment facility.

In addition to the active agent, the formulation can include other suitable polymers, e.g., to permit the resulting formulation to be used to form a microparticle. In a preferred embodiment, a polymer used in this method is biocompatible. A polymer is biocompatible if the polymer, and any degradation products of the polymer, such as metabolic products, are non-toxic to humans or animals, to whom the polymer was administered, and also present no significant deleterious or untoward effects on the-recipient's body, such as an immunological reaction at the injection site. Biocompatible polymers can be biodegradable polymers, non-biodegradable polymers, a blend thereof or copolymers thereof.

Suitable biocompatible, non-biodegradable polymers include, for instance, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof.

Suitable biocompatible, biodegradable polymers include, for example, poly(lactide)s, poly(glycolide)s, poly(lactide-co-glycolide)s, poly(lactic acid)s, poly(glycolic acid)s, polycarbonates, polyesteramides, polyanhydrides, poly(amino acids), polyorthoesters, polyacetals, polycyanoacrylates, polyetheresters, polycaprolactone, poly(dioxanone)s, poly(alkylene alkylate)s, polyurethanes, blends and copolymers thereof. Polymers comprising poly(lactides), copolymers of lactides and glycolides, blends thereof, or mixtures thereof are more preferred. Said polymers can be formed from monomers of a single isomeric type or a mixture of isomers.

A polymer used in this method can be blocked, unblocked or a blend of blocked and unblocked polymers. An unblocked polymer is as classically defined in the art, specifically having free carboxyl end groups. A blocked polymer is also as classically defined in the art, specifically having blocked carboxyl end groups. Generally, the blocking group is derived from the initiator of the polymerization reaction and is typically an alkyl radical.

In certain embodiments, the subject formulations are prepared by lyophilization. The simplest form of lyophilizer would consist of a vacuum chamber into which wet sample material could be placed, together with a means of removing water vapor so as to freeze the sample by evaporative cooling and freezing and then maintain the water-vapor pressure below the triple-point pressure.

### EXAMPLE 1

Release of bovine serum albumin (BSA) was measured up to 811 hours from samples of lyophilized protein precipitated from an alcohol/aqueous solution. This example briefly describes sample preparation and analytical methodology and presents results showing controlled release of BSA. The release is affected by the specific alcohol used, the buffer used, and the particle size of the precipitated and lyophilized protein.

Solutions of BSA (USB, Amersham Life Sciences, Cat. No. 10868) at 5% (w/w) were prepared in 0.01 M acetate buffer using an equivalent volume of 0.005 M sodium acetate and 0.005 M acetic acid. The pH was approximately 5. The alcohol n-propanol was added to a concentration of 40% (v/v). After, overnight equilibration at room temperature, the supernatant was removed and the precipitate frozen at −20 ° C. and brought to −70 ° C. before lyophilization. The surface upon which the vials were placed and the lyophilizer chamber was precooled to maintain the samples frozen during the lyophilization procedure. The sample was lyophilized for 5 hours. The time of lyophilization can be longer or shorter depending on the volume to be lyophilized. The lyophilized sample was divided into several pieces with a spatula. The pieces were divided into small particles by crushing the pieces against the wall and bottom of the glass vial. The larger masses and small crushed particles were weighed so that 5 to 10 mg of the masses and the crushed particles were placed into separate 1.5 ml conical polypropylene tubes, then 1 ml of phosphate buffered saline was added. The masses or particles were disbursed into the liquid. One hour after disbursing the samples, the contents of the tubes were mixed again and then the tubes centrifuged for 5 minutes at 5,000 rpm (Eppendorf Centrifuge, Model No. 5415). A sample of 0.1 ml was removed for assay and replaced with 0.1 ml of PBS. This procedure was repeated to take samples at 65 hours. At 98 hours and each time point thereafter, the full volume of release medium was removed and replaced with a fresh 1 ml of PBS.

Samples were analyzed for protein content with the microassay procedure for microtiter plates (Bio-Rad protein assay, based on the method of Bradford; Coomassie Brilliant Blue Dye, Cat. No. 500-0006) with 96 well microtiter plates. Standards contained 5 to 60 μg/ml of BSA. Standards and samples were added to the wells in a volume of 0.16 ml first, then 40 μl of dye was added to each well with mixing before reading the absorbance at 630 nm. Standard curves were constructed from absorbances corrected for the blank values in the absence of added protein (BSA). Protein concentrations of the-samples were calculated from the standard curve that was based on the same lot of BSA and prepared on the basis of weight of BSA to total volume (w/v). The values for the protein released at various times were adjusted by determining differences in the protein concentration of the lyophilized BSA that was weighed and placed in solution from the BSA taken directly from the bottle of the commercial supplier (USB, Amersham Life Sciences, Cat. No. 10868) and placed in solution.

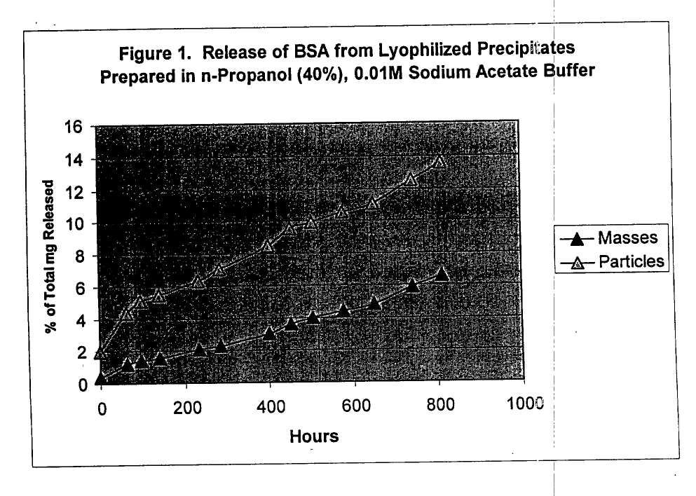

The results of the controlled release are shown in FIG. 1 [nP, represents n-propanol]. As can be seen, there is little or no burst effect and the release is essentially linear. The smaller particles with a large surface area to mass ratio release at a faster rate. There appears to be a slightly faster rate of release during the first hours of release (FIG. 1). This faster release rate can be eliminated by preincubating the samples in medium prior to use.

### EXAMPLE 2

Release of BSA was measured up to 811 hours from samples of lyophilized protein precipitated from alcohol/aqueous solution. This example briefly describes sample preparation and analytical methodology and presents results showing controlled release of BSA. The release is affected by the specific alcohol used, the buffer used, and the particle size of the precipitated and lyophilized protein.

Solutions of BSA (USB, Amersham Life Sciences, Cat. No. 10868) at 5% (w/w) were prepared in 0.1 M acetate buffer using an equivalent volume of 0.05 M sodium acetate and 0.05 M acetic acid. The pH was approximately 5. The alcohol n-propanol was added to a concentration of 50% (v/v). After overnight equilibration at room temperature, the supernatant was removed and the precipitate frozen at −20 ° C. and brought to −70 ° C. before lyophilization. The surface upon which the vials were placed and the lyophilizer chamber was precooled to maintain the samples frozen during the lyophilization procedure. The sample was lyophilized for 5 hours. The time of lyophilization can be longer or shorter depending on the volume to be lyophilized. The lyophilized sample was divided into several pieces with a spatula. The pieces were divided into small particles by crushing the pieces against the wall and bottom of the glass vial. The larger masses and small crushed particles were weighed so that 5 to 10 mg of the masses and the crushed particles were placed into separate 1.5 ml conical polypropylene tubes, then 1 ml of phosphate buffered saline was added. The masses or particles were disbursed into the liquid. One hour after disbursing the samples, the contents of the tubes were mixed again and then the tubes centrifuged for 5 minutes at 5,000 rpm (Eppendorf Centrifuge, Model No. 5415). A sample of 0.1 ml was removed for assay and replaced with 0.1 ml of PBS. This procedure was repeated to take samples at 65 hours. At 98 hours and each time point thereafter, the full volume of release medium was removed and replaced with a fresh 1 ml of PBS.

Samples were analyzed for protein content with the microassay procedure for microtiter plates (Bio-Rad protein assay, based on the method of Bradford; Coomassie Brilliant Blue Dye, Cat. No. 500-0006) with 96 well microtiter plates. Standards contained 5 to 60 μg/ml of BSA. Standards and samples were added to the wells in a volume of 0.16 ml first, then 40 μl of dye was added to each well with mixing before reading the absorbance at 630 nm. Standard curves were constructed from absorbances corrected for the blank values in the absence of added protein (BSA). Protein concentrations of the samples were calculated from the standard curve that was based on the same lot of BSA and prepared on the basis of weight of BSA to total volume (w/v). The values for the protein released at various times were adjusted by determining differences in the protein concentration of the lyophilized BSA that was weighed and placed in solution from the BSA taken directly from the bottle of the commercial supplier (USB, Amersham Life Sciences, Cat. No. 10868) and placed in solution.

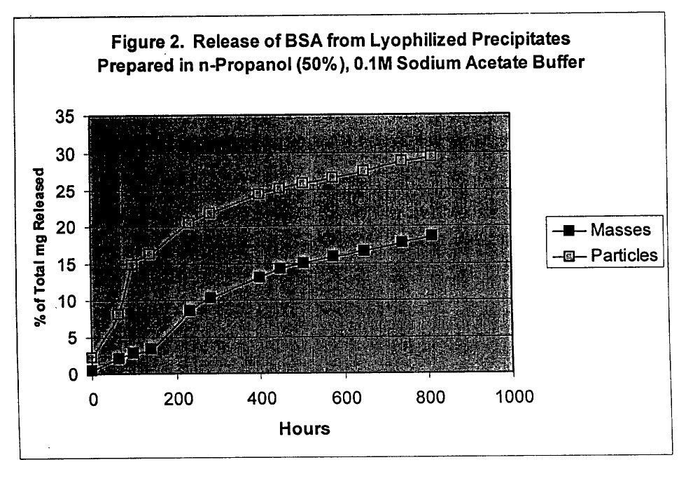

The results of the controlled release are shown in FIG. 2 [nP, represents n-propanol]. As can be seen, there is no burst effect and the release is essentially linear. The smaller particles with a large surface area to mass ratio release at a faster rate. There appears to be a slightly faster rate of release during the first hours of release (FIG. 2). This faster release rate can be eliminated by preincubating the samples in medium prior to use.

### EXAMPLE 3

Release of BSA was measured up to 811 hours from samples of lyophilized protein precipitated from alcohol/aqueous solution. This example briefly describes sample preparation and analytical methodology and presents results showing controlled release of BSA. The release is affected by the specific alcohol used, the buffer used, and the particle size of the precipitated and lyophilized protein.

Solutions of BSA (USB, Amersham Life Sciences, Cat. No. 10868) at 5% (w/w) were prepared in 0.01 M acetate buffer using an equivalent volume of 0.005 M sodium acetate and 0.005 M acetic acid. The pH was approximately 5. The t-butyl alcohol was added to a concentration of 40% (v/v). After overnight equilibration at room temperature, the supernatant was removed and the precipitate frozen at −20 ° C. and brought to −70 ° C. before lyophilization. The surface upon which the vials were placed and the lyophilizer chamber was precooled to maintain the samples frozen during the lyophilization procedure. The sample was lyophilized for 5 hours. The time of lyophilization can be longer or shorter depending on the volume to be lyophilized. The lyophilized sample was divided into several pieces with a spatula. The pieces were divided into small particles by crushing the pieces against the wall and bottom of the glass vial. The larger masses and small crushed particles were weighed so that 5 to 10 mg of the masses and the crushed particles were placed into separate 1.5 ml conical polypropylene tubes, then 1 ml of phosphate buffered saline was added. The masses or particles were disbursed into the liquid. One hour after disbursing the samples, the contents of the tubes were mixed again and then the tubes centrifuged for 5 minutes at 5,000 rpm (Eppendorf Centrifuge, Model No. 5415). A sample of 0.1 ml was removed for assay and replaced with 0.1 ml of PBS. This procedure was repeated to take samples at 65 hours. At 98 hours and each time point thereafter, the full volume of release medium was removed and replaced with a fresh 1 ml of PBS.

Samples were analyzed for protein content with the microassay procedure for microtiter plates (Bio-Rad protein assay, based on the method of Bradford; Coomassie Brilliant Blue Dye, Cat. No. 500-0006) with 96 well microtiter plates. Standards contained 5 to 60 μg/ml of BSA. Standards and samples were added to the wells in a volume of 0.16 ml first, then 40 μl of dye was added to each well with mixing before reading the absorbance at 630 nm. Standard curves were constructed from absorbances corrected for the blank values in the absence of added protein (BSA). Protein concentrations of the samples were calculated from the standard curve that was based on the same lot of BSA and prepared on the basis of weight of BSA to total volume (w/v). The values for the protein released at various times were adjusted by determining differences in the protein concentration of the lyophilized BSA that was weighed and placed in solution from the BSA taken directly from the bottle of the commercial supplier (USB, Amersham Life Sciences, Cat. No. 10868) and placed in solution.

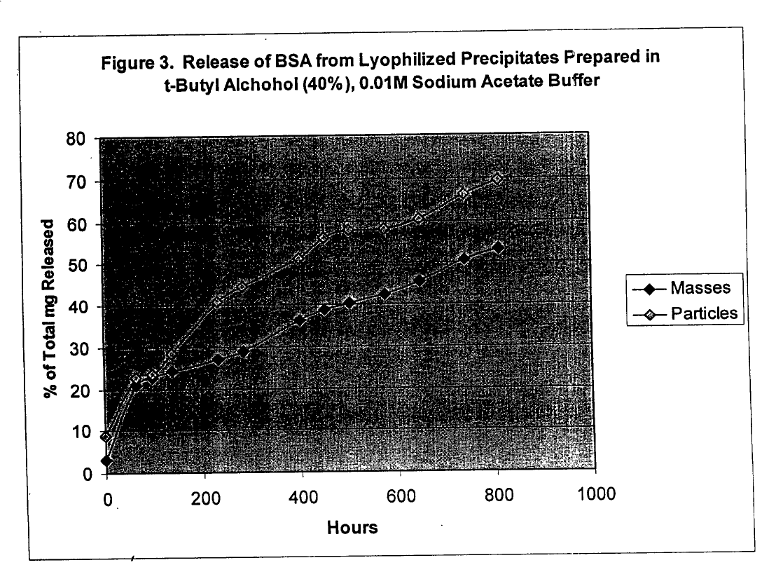

The results of the controlled release are shown in FIG. 3 [tBA, represents t-butyl alcohol]. As can be seen, there is no major burst effect and the release is essentially linear after the first hours. The smaller particles with a large surface area to mass ratio release at a faster rate. There appears to be a slightly faster rate of release during the first hours of release (FIG. 3). This faster release rate can be eliminated by preincubating the samples in medium prior to use.

### EXAMPLE 4

Release of BSA was measured up to 811 hours from samples of lyophilized protein precipitated from alcohol/aqueous solution. This example briefly describes sample preparation and analytical methodology and presents results showing controlled release of BSA. The release is affected by the specific alcohol used, the buffer used, and the particle size of the precipitated and lyophilized protein.

Solutions of BSA (USB, Amersham Life Sciences, Cat. No. 10868) at 5% (w/w) were prepared in 0.1 M acetate buffer using an equivalent volume of 0.05 M sodium acetate and 0.05 M acetic acid. The pH was approximately 5. The alcohol t-butyl alcohol was added to a concentration of 40% (v/v). After overnight equilibration at room temperature, the supernatant was removed and the precipitate frozen at −20 ° C. and brought to −70 ° C. before lyophilization. The surface upon which the vials were placed and the lyophilizer chamber was precooled to maintain the samples frozen during the lyophilization procedure. The sample was lyophilized for 5 hours. The time of lyophilization can be longer or shorter depending on the volume to be lyophilized. The lyophilized sample was divided into several pieces with a spatula. The pieces were divided into small particles by crushing the pieces against the wall and bottom of the glass vial. The larger masses and small crushed particles were weighed so that 5 to 10 mg of the masses and the crushed particles were placed into separate 1.5 ml conical polypropylene tubes, then 1 ml of phosphate buffered saline was added. The masses or particles were disbursed into the liquid. One hour after disbursing the samples, the contents of the tubes were mixed again and then the tubes centrifuged for 5 minutes at 5,000 rpm (Eppendorf Centrifuge, Model No. 5415). A sample of 0.1 ml was removed for assay and replaced with 0.1 ml of PBS. This procedure was repeated to take samples at 65 hours. At 98 hours and each time point thereafter, the full volume of release medium was removed and replaced with a fresh 1 ml of PBS.

Samples were analyzed for protein content with the microassay procedure for microtiter plates (Bio-Rad protein assay, based on the method of Bradford; Coomassie Brilliant Blue Dye,Cat. No. 500-0006) with 96 well microtiter plates. Standards contained 5 to 60 μg/ml of BSA. Standards and samples were added to the wells in a volume of 0.16 ml first, then 40 μl of dye was added to each well with mixing before reading the absorbance at 630 nm. Standard curves were constructed from absorbances corrected for the blank values in the absence of added protein (BSA). Protein concentrations of the samples were calculated from the standard curve that was based on the same lot of BSA and prepared on the basis of weight of BSA to total volume (w/v). The values for the protein released at various times were adjusted by determining differences in the protein concentration of the lyophilized BSA that was weighed and placed in solution from the BSA taken directly from the bottle of the commercial supplier (USB, Amersham Life Sciences, Cat. No. 10868) and placed in solution.

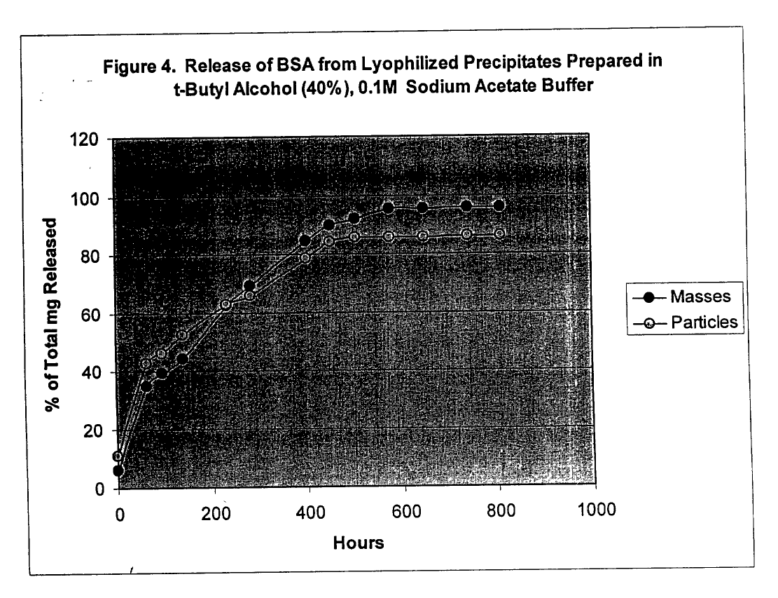

The results of the controlled release are shown in FIG. 4 [tBA, represents t-butyl alcohol]. As can be seen, there is no major burst effect and the release is essentially linear after the first hours. The smaller particles with a large surface area to mass ratio release at a faster rate. There appears to be a slightly faster rate of release during the first hours of release (FIG. 4). This faster release rate can be eliminated by preincubating the samples in medium prior to use.

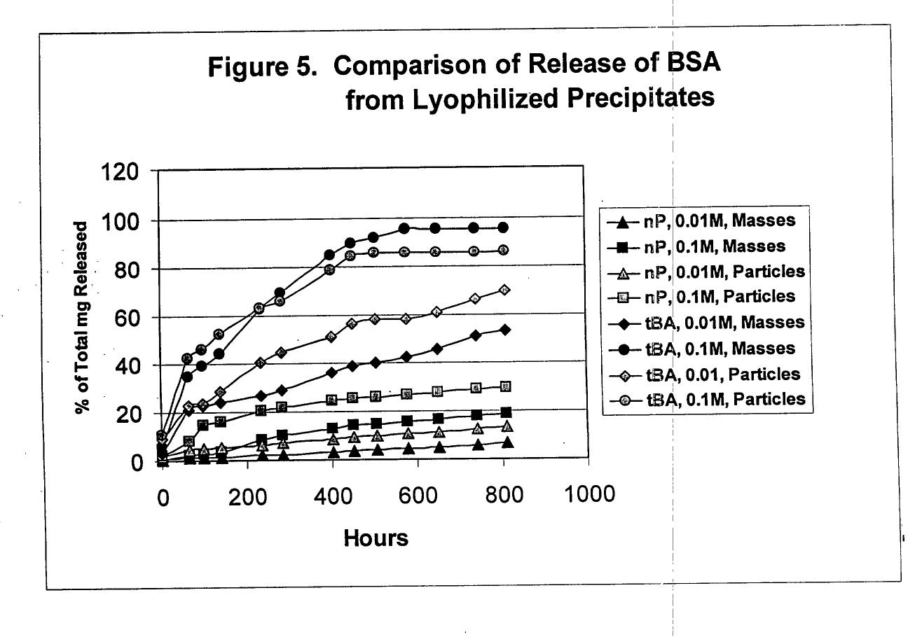

Comparison of the Release Data. A comparison of the release kinetics for all the samples are shown together on a single chart (FIG. 5). It can be seen that the various samples have release kinetics that will last for a wide variety of periods: from 500 hours (21 days) to about 10,000 hours (over 1 year). Combinations of the samples can produce release kinetics with a variety of release rates at different times. The small particles exhibited faster release rates except for the most rapidly releasing preparation (FIG. 5; FIG. 4; 0.1 M acetate; t-butyl alcohol, 40%). The results demonstrate that salt concentrations and the type of alcohol can modify the release rates extensively.

General Materials and Methods for Examples 5-13

(i) Materials

Bovine Serum Albumin (Cat. #10868, lot # 107331, USB)

Human Serum Albumin (Cat. #10878, lot #103077, USB)

Albumin (Human) 25% Solution: Immuno-U.S., Inc. (NDC 64193-228-05, lot # 628808)

Albumin (Human) 25% Solution: Alpha Therapeutic (Cat # 521302, lot # NG9856A)

Interferon-α001 (PBL) 0.94 mg/ml in Tris Buffer [see also U.S. Pat. Nos. 5,789,551, 5,869,293, 6,001,589, 6,299,870, 6,300,474]

Interferon-α012 (PBL) 1.38 mg/ml in Tris Buffer

Tris Buffer (20 mm Tris, 200 mm NaCl, 6% glycerol, pH 7-8)

Interferon ELISA (PBL product #41110)

PBS (Dulbecco's Phosphate Buffered Saline, Cat. #8537, Sigma Chemical Co., or Cat. #14198-144, Gibco-BRL)

(ii) Methods

Protein precipitation. Proteins were precipitated at ambient temperature (about 24° C.) by one of two basic procedures: the organic addition method or the acid addition method. With the organic addition method, the protein solution was prepared in aqueous solution and an organic component added to precipitate the protein. (Alternatively, an aqueous solution containing protein can be added to the organic solution.) For the acid addition method, a portion of the organic component was added to the protein solution under conditions that do not precipitate the protein. Precipitation was initiated by adding an acidified solution concurrent with or after addition of organic components to the protein solution. Unless otherwise stated in the legends, deionized water was used to dilute formulation reagents. HSA stock solutions were made by diluting 25% source material to 1% final concentration, and data presented were obtained using Immuno-U.S. Human Serum Albumin.

Adjustment of pH. Because organic solvent hinders the ability to accurately measure pH, the pH specified for any formulation refers to the pH of the (aqueous) solution prior to addition of the organic component. In the case of the organic addition method, the pH of an aqueous protein solution was adjusted to the desired pH just prior to adding the organic component. To make the same formulation by the acid addition method, an equivalent amount of acid was added in the final step rather than prior to addition of the organic solvent.

Maturation procedures. The maturation period began after addition of the final formulation component to initiate precipitation and ended when centrifugation was initiated to separate precipitate from supernatant. The release properties of the precipitate depend on the maturation time as well as the conditions of the formulation during this period. Temperature was ambient, about 24° C. unless otherwise noted. Formulations were mixed by vessel rotation, stirred in tubes or in vials containing a magnetic stir-bar, or mixed initially and left undisturbed. In addition, during the maturation period some formulations were drawn through a syringe needle one to three times toward the end of the maturation period.

Wash procedures. The first steps in washing precipitates were to 1) separate the precipitate from supernatant by centrifugation, 2) remove as much supernatant as possible without disturbing the precipitate, and 3) re-suspend the precipitate in PBS/0.01% thimerosal. Precipitates were harvested and washed (PBS/0.01% thimerosal) once or twice by centrifugation for 2-5 minutes at 3,000 to 15,000 rpm in a Beckman or Eppendorf microcentrifuge. A sample of the harvested supernatant was diluted 10-fold in PBS/0.01% thimerosal to prevent (through dilution of organic and acid) further precipitation of protein in the diluted supernatant. If the release experiment was to begin immediately, the last harvested wash sample was labeled as the zero time sample and the resuspended preparations placed in an incubator at 37 ° C. at which temperature release was measured for all samples. Alternatively, the sample could be lyophilized without resuspension after initial harvest or after wash cycles.

Lyophilization. Precipitates to be lyophilized without washing were cooled to 0-4° C., then sequentially at −20° C., −70° C., and −135° C., at least 15 minutes at each temperature. Precipitates to be lyophilized after washing with PBS/0.01% thimerosal were frozen only at −20° C. Formulations were lyophilized in a Virtis Freezemobile 6 equipped with a Unitop 100 SM Bulk/Stoppering Chamber. The lyophilizer shelf was pre-cooled with dry ice before transferring vials from the freezer to the shelf. Vials were lyophilized for 2-5 hours at <400 mTorr.

Release measurements. Sufficient PBS to make a total volume of 1 ml of release medium (PBS/0.01% thimerosal) was added to the washed and/or lyophilized precipitates. Each precipitate was suspended in release medium (PBS/0.01% thimerosal) before placing the release sample in a 37° C. incubator to begin measuring release of the proteins. At selected time intervals, tubes containing the samples with the release medium were removed from the incubator and centrifuged for 2-5 minutes at 3,000 to 15,000 rpm. The majority of the medium containing the released protein in the supernatant, usually about 0.9 ml, was removed and replaced with an equal volume of fresh PBS/0.01% thimerosal.

Sample analysis. Albumin samples were assayed as is or diluted with PBS/0.01% thimerosal to the range of the Bio-Rad Protein Assay (Bio-Rad Labs). Stock solutions diluted from the source albumin raw material in the formulations were used as assay standards. Interferon samples, as is or diluted with PBS/0.01% thimerosal, were assayed by ELISA (PBL Biomedical Laboratories, product # 41110).

Calculations. The cumulative quantity of analyte released at each sample time was calculated by adding the amount released in the n^{th} sample to the sum of the quantities released in the previous samples. The quantity released in the n^{th} sample was corrected for the residual quantity left in the tube from the previous sample since typically 0.9 ml of the total volume of 1.0 ml was collected at each sample interval. Cumulative quantities released were plotted as the mass released or as a percentage of the calculated total analyte present in the precipitate at the start of incubation at 37° C. (start of the release). The total analyte present in the precipitates at the start of the release was calculated by subtracting the quantity of analyte recovered in the supernatant and wash samples from the original amount of analyte added to the formulation.

### EXAMPLE 5

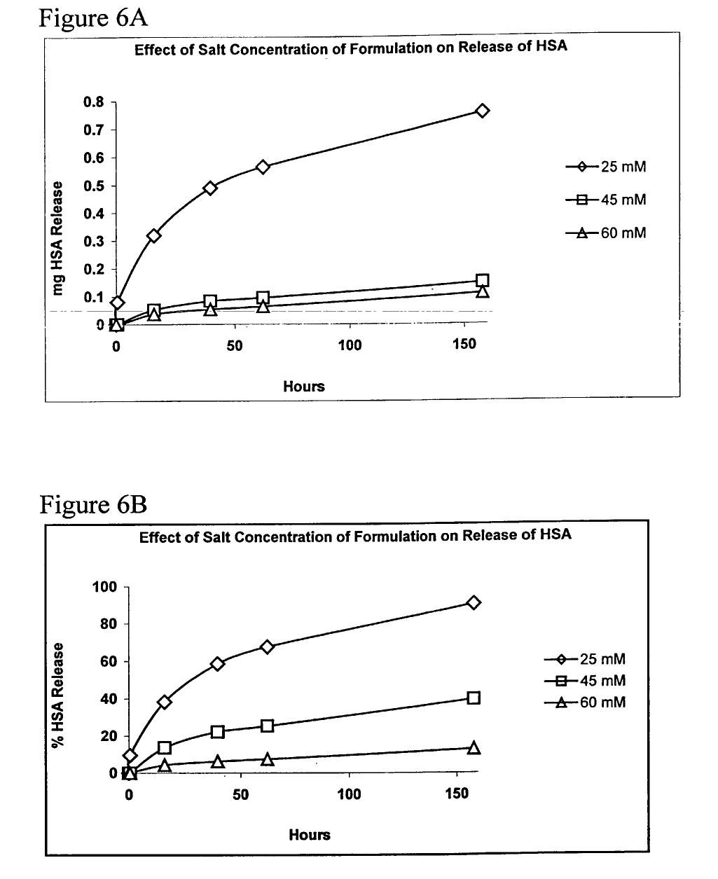

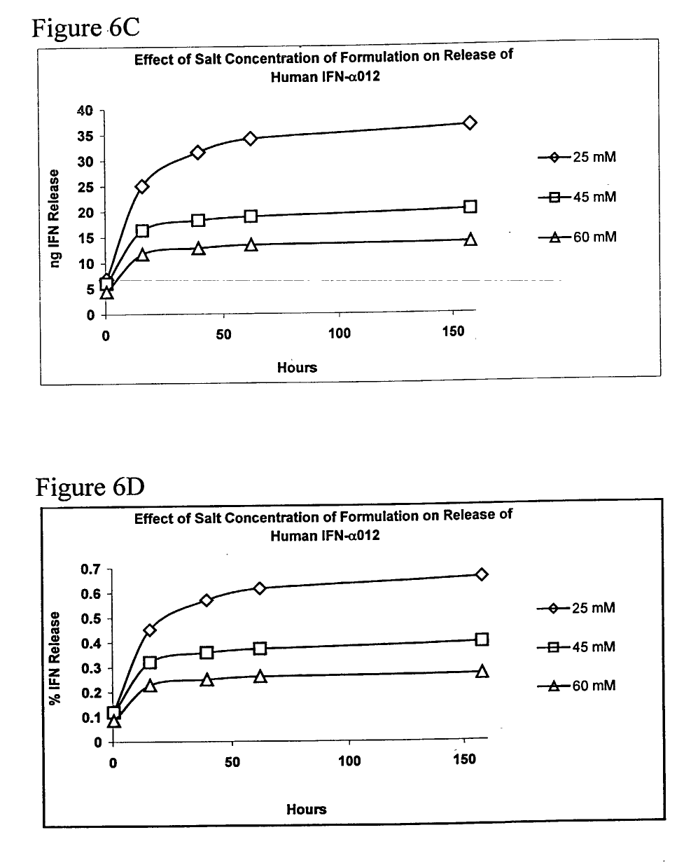

As an embodiment of the sustained release, the release of HSA and human IFN-α012 as a function of sodium acetate concentration was evaluated as shown in FIG. 6. Solution I consisted of 9.0 mg of HSA (Immuno-U.S.) and 10 μg of IFN-α012 in 40% (w/w) n-propanol (0.364 g n-propanol) in H₂O for a total Weight of 0.91 g. The various Solution II compositions consisted of various quantities of sodium acetate (1 M, pH 6.3) and deionized water and 0.040 g n-propanol to make solutions of 40% n-propanol and 250, 450, and 600 mM final sodium acetate concentrations with a total volume of 0.10 g. Solution II (0.10 g) was added to Solution I (0.91 g) with stirring to yield a final 1.01 g of each formulation. The final 1.01 g formulations containing 40% n-propanol and 25, 45, and 60 mM concentrations of sodium acetate were stirred in 2 ml glass vials for 6 hours at 24° C. and passed through 25G syringe needles just prior to separating supernatants from precipitates. The quantity of HSA and IFN-α012 in-washed precipitates was determined as described in Materials and Methods. Release was performed in PBS/0.01% thimerosal. As can be seen the early burst phase of the sustained release and the rate of release of HSA and human IFN-α012 can be altered by the sodium acetate concentration. Higher sodium acetate concentration decreased the burst rate (0-24 hour period) extensively and decrease the rate of release of the HSA and human IFN-α012 (FIG. 6A-D). Release continued after analysis period of about 7 days. The burst phase for release of human IFN-α012 was especially sensitive to the sodium acetate concentration. The release was monitored for about 160 hours (over six days).

### EXAMPLE 6

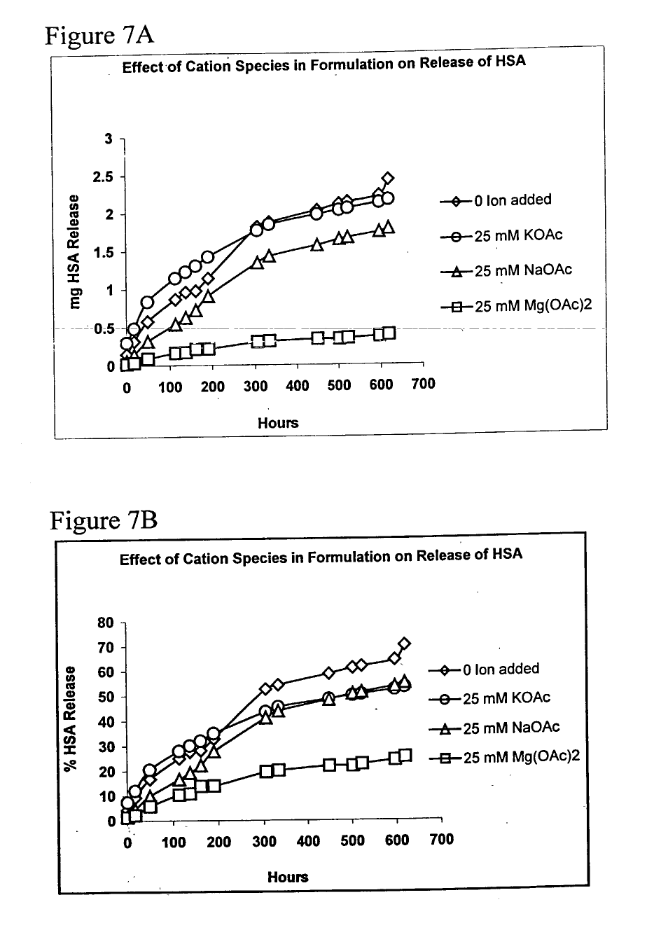

Effect of cation species in formulation on release of HSA is shown in FIG. 7A,B. Solution I consisted of 8.1 mg of HSA (Immuno-U.S.) in 40% (w/w) n-propanol in deionized water in a total volume of 0.91 ml. The various Solution II compositions consisted of adding none or 0.025 ml of various salt stocks (each at 1 M cation concentration, pH 6.3) to deionized water followed by n-propanol to make solutions 40% (w/w) n-propanol and 250 mM final cation concentration in a total volume of 0.10 ml. Solution II (0.10 ml) was added to 0.91 ml of Solution I with stirring to give a final 1.01 ml formulation having 40% (w/w) n-propanol. The final 1.01 ml formulations containing 40% n-propanol and no or 25 mM concentrations of potassium, sodium or magnesium acetate were stirred in 2 ml glass vials for 6 hours at 24 ° C. prior to separating supernatants from precipitates. The quantity of HSA in washed precipitates was determined as described in Materials and Methods. Release was performed in PBS/0.01% thimerosal. The burst rate in the first 24 hours was reduced substantially by sodium and even further by magnesium in the formulation. Furthermore, the release rate can be increased or reduced by use of the various acetates. Extended release rates of over 25 days (over 600 hours) were achieved with all these formulations. Release was projected to continue beyond the time measured by the graphs (FIG. 7A,B). The release was monitored for over 600 hours or 25 days.

### EXAMPLE 7

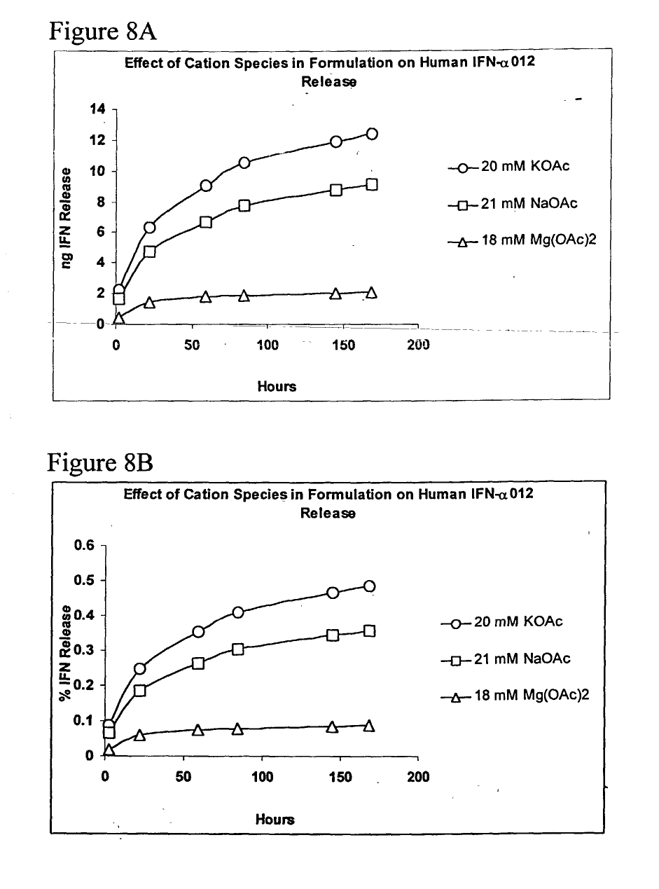

Effect of cation species in formulation on release of human IFN-α012 is shown in FIG. 8A,B. Solution I consisted of 45 mg of HSA (Immuno-U.S.) and 5.44 μg IFN-α012 in 40% (w/w) n-propanol in deionized water in a total volume of 4.55 ml. The various Solution II compositions consisted of adding 36 μl of 0.1 M acetic acid (to compensate for the buffer capacity of the HSA solution) and 0.250 g of potassium, sodium or magnesium acetate solution (each at pH 6.3) to 0.314 g of deionized water and 0.400 g of n-propanol to make solutions of 40% (w/w) n-propanol and 250 mM final acetate concentration in a total weight of 1 g. The potassium acetate solution was made with 0.980 g potassium acetate, 10.061g water and 0.274 ml 1 M acetic acid. The sodium acetate solution was made with 0.823 g sodium acetate, 10.056 g water and 0.245 ml 1 M acetic acid. The magnesium acetate solution was made with 2.144 g magnesium acetate, 10 g water and 0.200 ml 1 M acetic acid. Solution II (0.50 ml) was added to 4.55 ml of Solution I with stirring to give a final 5.05 ml formulation having 40% (w/w) n-propanol. The final formulations were stirred in 50 ml conical tubes for 6 hours at 24° C., the precipitates washed with 5 ml of PBS/0.01% thimerosal, then suspended in 5 ml PBS/0.01% thimerosal, then split into two individual 2.5 ml samples prior to separating supernatants from precipitates. Release data are from the precipitates from one 2.5 ml portion of the formulation. The salt concentrations in the formulations were 21 mM NaOAc, 20 mM KOAc and 18 mM Mg(OAc)₂ in the respective solutions. The quantity of IFN-α012 in washed precipitates was determined as described in Materials and Methods. Release was performed in PBS/0.01% thimerosal. The burst rate can be reduced extensively from potassium to sodium and to magnesium acetate in that order (FIG. 8). In addition, the overall rate of release can be modulated by these salts: the rate of release of IFN-α012 is fastest with potassium acetate, less with sodium acetate and slowest with magnesium acetate (FIG. 8). The release was monitored for about 170 hours or seven days.

### EXAMPLE 8

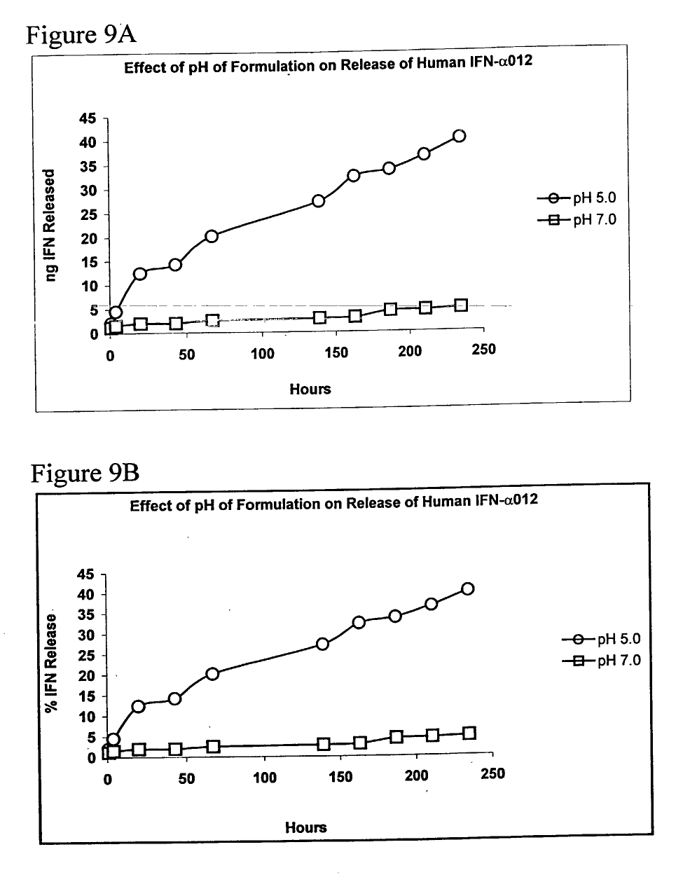

Effect of pH of formulation on release of human IFN-α012 is shown in FIG. 9. Acetic acid (0.1 M) was used to adjust 5% HSA stock solutions to pH 5.0 or pH 7.0. Solution I consisted of 10 mg of HSA (Alpha Therapeutic) from either pH 5.0 or pH 7.0 HSA stock solutions, 6.83 pg IFN-α012 and additional water to a total weight of 0.6 g. The final formulations were prepared by adding 0.4 g of n-propanol to Solution I with stirring to yield a concentration of 40% (w/w) n-propanol. Final 1 g formulations were stirred in 2 ml glass vials for 24 hours at 24° C. prior to separating supernatants from precipitates. The quantity of IFN-α012 in washed precipitates was determined as described in Materials and Methods. Release was performed in PBS/0.01% thimerosal. The burst was modest at both pH 5.0 and pH 7.0 and was remarkably approaching linearity at both pH values (FIG. 9). The lower pH increased the rate of release extensively. Relatively little or no overall burst effect was evident. The release was monitored for about 240 hours or ten days.

### EXAMPLE 9

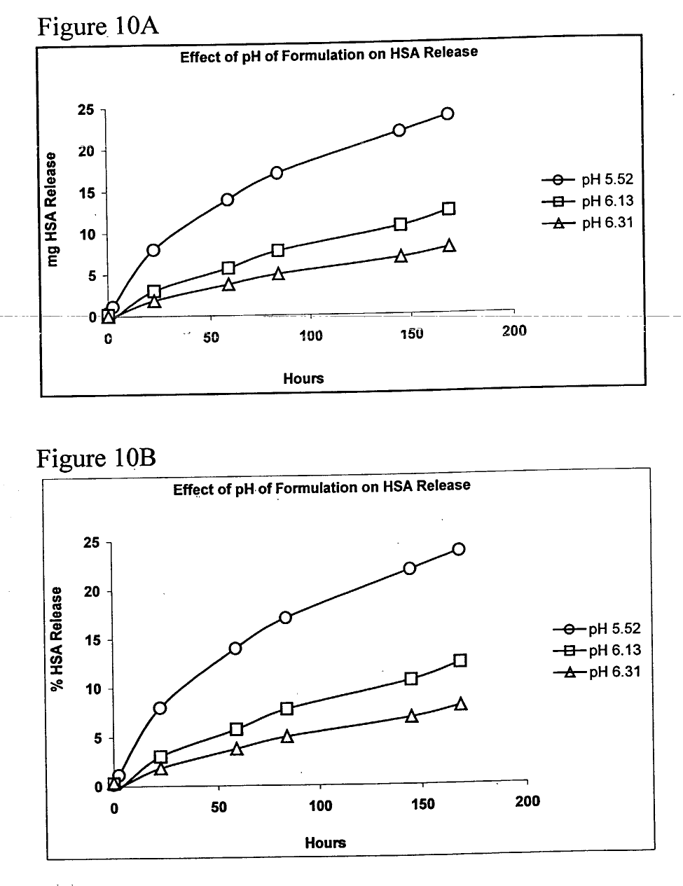

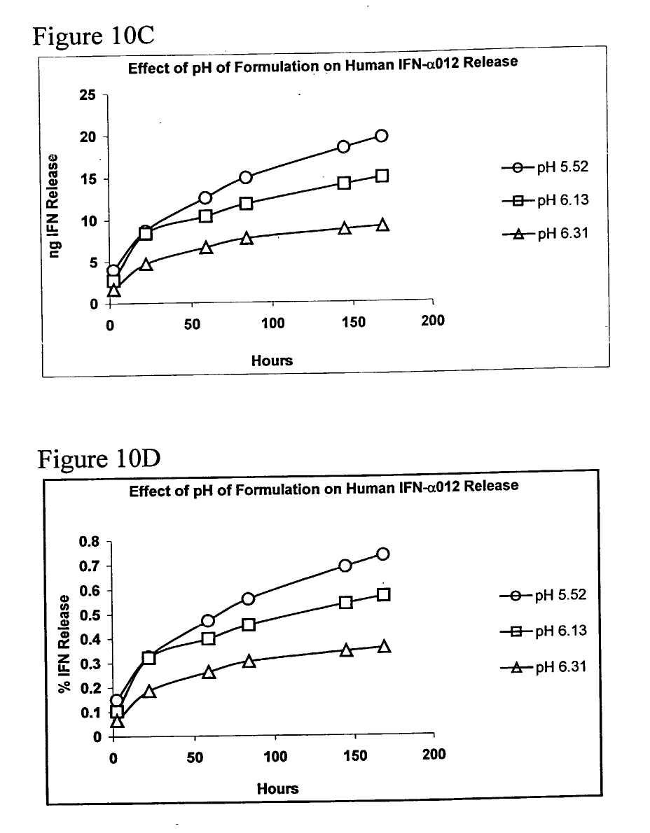

Effect of pH of formulation on release of HSA and human IFN-α012 is shown (FIG. 10A-D). Solution I consisted of 45 mg of HSA (Immuno-U.S.) and 5.44 μg IFN-α012 in 40% (w/w) n-propanol in deionized water in a total volume of 4.55 ml. Solution II compositions were prepared as follows. Solution IIa: 1.55 ml of 1 M acetic acid was added to 0.82 g anhydrous sodium acetate and 10 g deionized water to adjust pH of this Solution A to 5.52; then 0.036 ml of 0.1 M acetic acid was added to 0.250 g of Solution A to compensate for the buffer capacity of the HSA solution deionized water added to bring the total weight to 0.600 g; hen 0.400 g of n-propanol was added to make a final solution of 40% (w/w) n-propanol in a total weight of 1.00 g. Solution IIb: 0.40 ml of 1 M acetic acid was added to 0.82 g anhydrous sodium acetate and 10 g of deionized water to adjust pH of this Solution B to 6.13; then 0.036 ml of 0.1 M acetic acid was added to 0.250 g of Solution B to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. Solution IIc: 0.245 ml of 1 M acetic acid was added to 0.823 g anhydrous sodium acetate and 10.056 g deionized water to adjust pH of this Solution C to 6.31; then 0.036 ml of 0.1 M acetic acid was added to 0.250 g of Solution C to compensate for the buffer capacity of the HSA solution; deionized water was then added to bring the total weight to 0.600 g; then 0.400 g of n-propanol was added to make a final solution of 40% (w/w) n-propanol in a total weight of 1.00 g. To prepare the final formulations, 0.50 ml from Solutions Ia, IIb, or IIc was added to 4.55 ml of Solution I with stirring to yield three 5.05 ml formulations having 40% (w/w) n-propanol and pH 5.52, pH 6.13 or pH 6.31, respectively. Final formulations were stirred in 50 ml conical tubes for 6 hours at 24° C., then split into two individual 2.52 ml samples prior to separating supernatants from precipitates. Release data is from one 2.52 ml portion of the formulation. The amount of IFN-α012 in washed precipitates was determined as described in Materials and Methods. Release was performed in PBS/0.01% thimerosal. The overall burst was minimal at all pH values (pH 5.52, pH 6.13 and pH 6.31 (FIG. 10A,B) for HSA, but slightly greater for human IFN-α012 (FIG. 10C,D). The rate of release of both HSA and human IFN-α012 was increased by lowering the pH in all cases (FIG. 10A-D) as also shown in FIG. 9. Of note is that small changes in the pH can modulate the rate of release and that overall changes in release are the same for HSA and IFN-α012.

### EXAMPLE 10

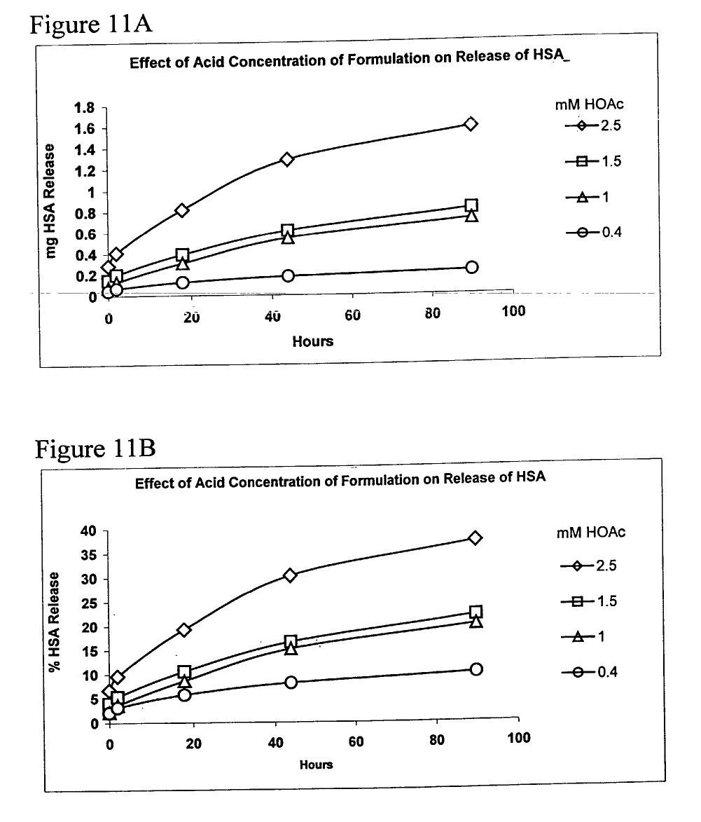

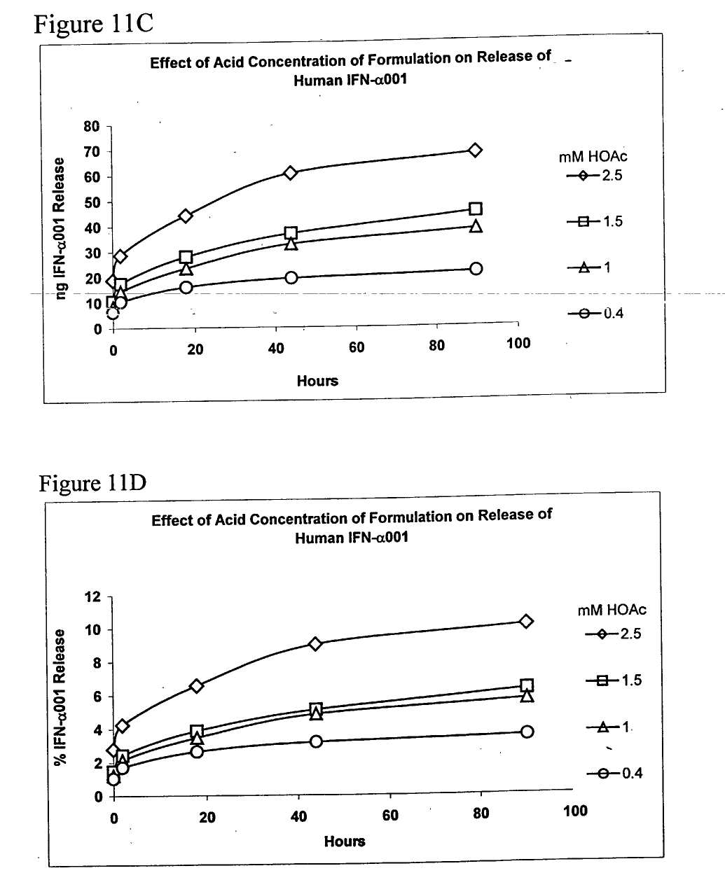

Effect of acid concentration of formulation on release of HSA and human IFN-α001 from precipitates formed in the presence of 25 mM sodium acetate is shown in FIG. 11. Solution I consisted of 8.1 mg of HSA (Immuno-U.S.) and 0.92 μg IFN-α001 in 40% (w/w) n-propanol in deionized water in a total volume of 0.9 ml. Several Solution II formulations, Ia, IIb, IIc and IId, were prepared consisting of 0.004, 0.010, 0.015 and 0.025 ml of 0.1 M acetic acid, respectively, in 40% (w/w) n-propanol in deionized water. Solution III consisted of 1 M sodium acetate and 40% (w/w) n-propanol in deionized water in a total volume of 0.025 ml. Several Solution IV formulations, IVa, IVb, IVc and IVd, were prepared consisting of 0.071, 0.065, 0.060 and 0.050 ml of 40% (w/w) n-propanol, respectively, in deionized water. In preparing the final formulations, Solutions Ia, IIb, IIc and IId were matched with Solutions IVa, IVb, IVc and IVd, respectively. Solutions II, III and IV were mixed together then Solution I added rapidly to the mixture to give a final 1 ml formulation. This yielded a formulation having a final concentration of 25 mM sodium acetate, 40% (w/w) n-propanol and the final acetic acid concentrations indicated on the Figure. Formulations were stirred in 2 ml glass vials for 6 hours at 24° C. prior to separating supernatants from precipitates. After washing, precipitates were lyophilized 4 hours at <40,0 mTorr. The amount of HSA in washed precipitates was determined as described in Materials and Methods. Release was performed in PBS/0.01% thimerosal. The burst is increased by increased quantity of acetic acid comparable to the increase in burst on decrease of pH as seen in FIGS. 9 and 10. Furthermore, the rate of release increases with the quantity of acid also comparable to the increase in rate of release with decrease in pH as seen in FIGS. 9 and 10. The release of HSA and human IFN-α001 was monitored for about 90 hours (FIG. 11A-D).

### EXAMPLE 11

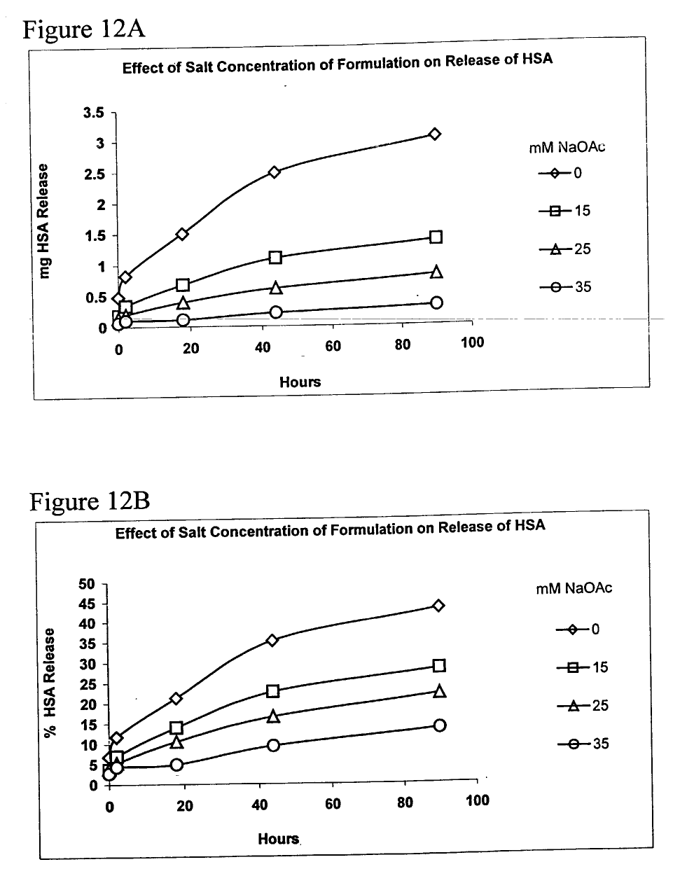

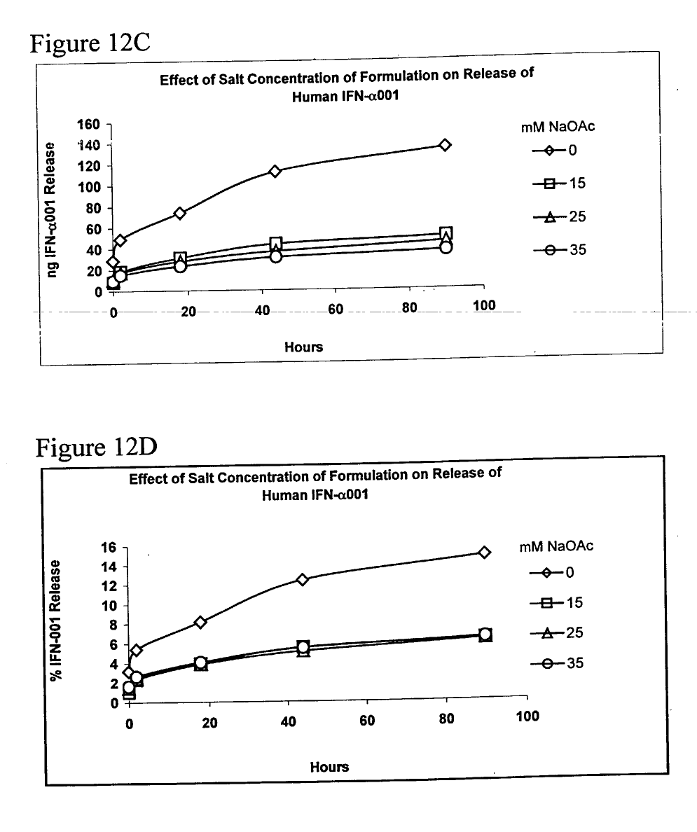

Effect of salt concentration of formulation on release of HSA and human IFN-α001 from precipitates formed in the presence of 1.5 mM acetic acid is shown in FIG. 12. Solution I consisted of 8.1 mg of HSA (Immuno-U.S.) and 0.92 μg IFN-α001 in 40% (w/w) n-propanol in deionized water in a total volume of 0.9 ml. Solution II consisted of 0.1 M acetic acid and 40% (w/w) n-propanol in deionized water in a total volume of 0.015 ml. Several Solution III formulations, IIIa, IIIb, IIIc and IIId, were prepared consisting of 0, 0.015, 0.025 and 0.035 ml of 1 M sodium acetate, respectively, in 40% (w/w) n-propanol in deionized water. Several Solution IV formulations, IVa, IVb, IVc and IVd, were prepared consisting of 0.085, 0.070, 0.060 and 0.050 ml of 40% (w/w) n-propanol, respectively, in deionized water. In preparing the final formulations, Solutions IIIa, IIIb, IIIc and IIId were matched with Solutions IVa, IVb, IVc and IVd, respectively. Solutions II, III and IV were mixed together then Solution I added rapidly to the mixture to give a final 1 ml formulation. This yielded a final concentration of 1.5 mM acetic acid, 40% (w/w) n-propanol (w/w) and the final sodium concentrations indicated on the Figure. Formulations were stirred in 2 ml glass vials for 6 hours at 24 ° C. prior to separating supernatants from precipitates. After washing, precipitates were lyophilized 4 hours at <400 mTorr. The amounts of HSA and IFN-α001 in washed precipitates were determined as described in Materials and Methods. Release was performed in PBS/0.01% thimerosal. Increased salt concentration minimizes the burst and reduces the rate of release of HSA (FIG. 12A, B) and IFN-α001 (FIG. 12C, D). Much of the burst can be eliminated by sodium acetate concentrations above 15 mM. The release was monitored for about 90 hours.

### EXAMPLE 12

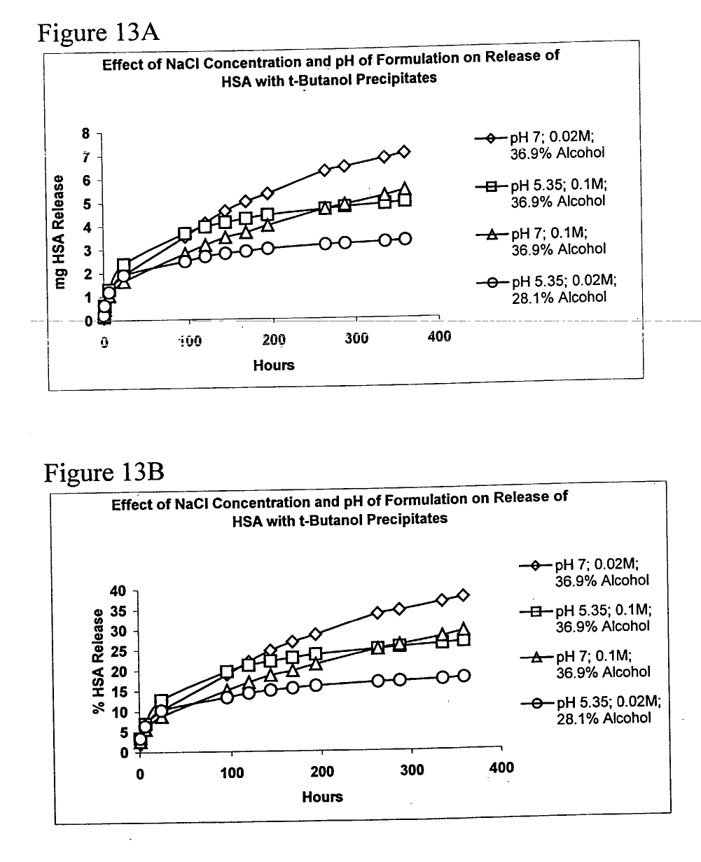

Effect of salt concentration and pH of formulation on release of HSA with tertiary butanol precipitates is shown in FIG. 13. Acetic acid (0.1 M) was used to adjust 5% HSA stock solutions to pH 5.35 or 7.0. Solution I consisted of 18.0 mg of HSA (Alpha Therapeutic) from the pH 5.35 or pH 7.0 5% stock solution, 1.0 μg IFN-α012 and deionized water bringing the total solution weight to 0.375 g. To prepare Solutions Ia and IIb with NaCl concentrations of 0.02 M and 0.1 M, respectively, sufficient deionized water was added to 0.021 and 0.0043 ml of a 3.75 M NaCl solution to bring the total weight of each solution to 0.425 g. Both pH 5.35 and pH 7.0 variants of Solution I (0.375 g) were added to Solutions IIa and IIb to yield 0.80 g of the various combinations of pH and NaCl concentration as shown in the Figure prior to the addition of 0.31 or 0.47 g of tert-butyl alcohol to yield 28.1% and 36.9% (w/w) tert-butyl alcohol (see summary of the chart legends). Final 1.11-1.27 g formulations were stirred in 2 ml glass vials for 24 hours at 24° C. prior to separating supernatants from precipitates. The amount of HSA in washed precipitates was determined as described in Materials and Methods. Release was performed in PBS/0.01% thimerosal. The pH had very little effect on the burst in the formulations with tertiary butyl alcohol (FIG. 13A and B). Furthermore, the rate of release of HSA was decreased by decrease in pH in. contrast to the formulations with n-propanol (FIGS. 9 and 10). Nevertheless, the overall rate of release of HSA over the 350 hours of monitoring (FIG. 13). The release rates were more near linearity at pH 7.0 than at pH 5.35.

### EXAMPLE 13

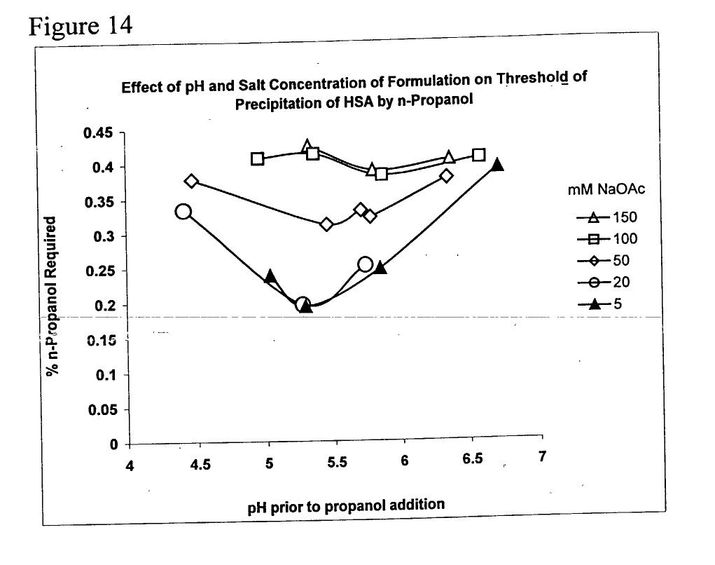

Effect of pH and salt concentration of formulation on threshold of precipitation of HSA by n-propanol is shown in FIG. 14. An 11% (w/w) HSA (USB) solution was dialyzed 3 times for 6 hours each time against 2 L deionized H₂O in a Pierce Slide_alyzer (15 ml capacity, No. 66410, lot # BJ44820B). The final concentration was analyzed by spectrophotometry at 280 nm to be 8.28% (w/w). This solution was diluted to 4% (w/w) with deionized water. Amounts (0.9 g) of 4% HSA were weighed into 2 ml glass vials. Sodium acetate (1 M), acetic acid (1 M), sodium hydroxide (1 M), and water were added in various combinations in a total weight of 0.1 g to yield the final sodium concentrations and pH values measured in 1 g formulations as shown in the Figure. Subsequently, n-propanol was added in about 50 μl increments with stirring, and the point at which initial precipitates were stable (did not re-dissolve with stirring within 5 minutes) was recorded. Connected data points indicate equivalent sodium concentrations at various pH and n-propanol (w/w) concentrations. The threshold of precipitation of HSA can be modified greatly by the sodium acetate concentration. At low sodium acetate concentrations, the least level of n-propanol is required to initiate precipitation of the HSA. These data (FIG. 14) provide general approaches to modulate the formulations.

### EXAMPLE 14

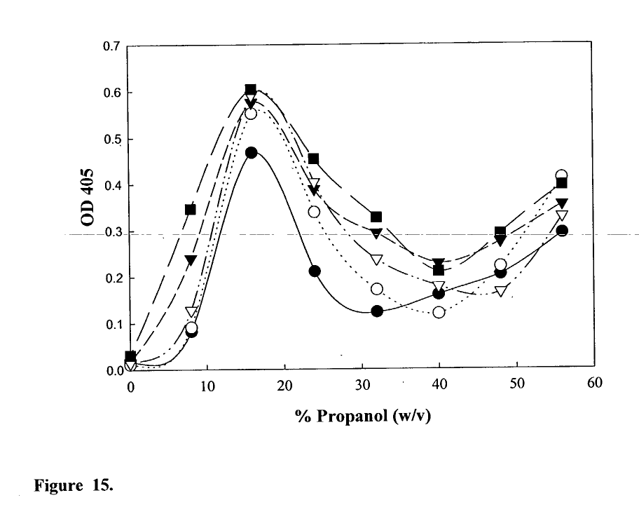

Effect of n-propanol concentration on the formation of human IFN-α001 particles at various pHs and buffers is shown in FIG. 15. Five different buffers were tested for this purpose: (1) 25 mM NaOAc pH 5.2; (2) 25 mM NaOAc pH 6.2; (3) 25 mM NaOAc pH 7.6; (4) 25 mM NaPO₄; (5) 25 mM Tris HCl. The final concentration of human IFN-α001 was 0.156 mg/ml with 2.2 mM Tris HCl, 24 mM NaCl, and 0.5% glycerol. Formation of human IFN-α001 particles was monitored by increase of absorbance at 405 nm using a microplate aggregation assay as described below.

Microplate Aggregation Assay. 100 μl of sample consisting of protein for formulation, 50 mM buffer and other additives if applicable were placed in a 96 well microplate (Immulon 2 HB, Dynax Technologies). For formulations having an n-propanol concentration of 32% and below, 100 μl of a 2× concentrated water/n-propanol mixture was then added to initiate formulation. For the 40% n-propanol samples 100 μl of 100% n-propanol was added to the protein solution. For the 48 and 56% w/v n-propanol samples 100 μl of 100 percent n-propanol was added to 100 μl of protein buffer mixture containing 8% and 16% w/v n-propanol respectively. The plate was sealed with a clear plastic adhesive cover and aggregate formation was monitored by reading the absorbance of the samples in a microplate reader (Biotek Microreader EL311) at 405 nm at various time points.

The results from the experiment indicate that particle formation for human IFN-α001 and IFN-α012 (data not shown) is mildly dependent on the pH of the buffer or the buffer salt species. In addition, the particle formation curve for interferon is unexpectedly biphasic with a peak at 16% n-propanol and a second peak at or above 56% n-propanol.

### EXAMPLE 15

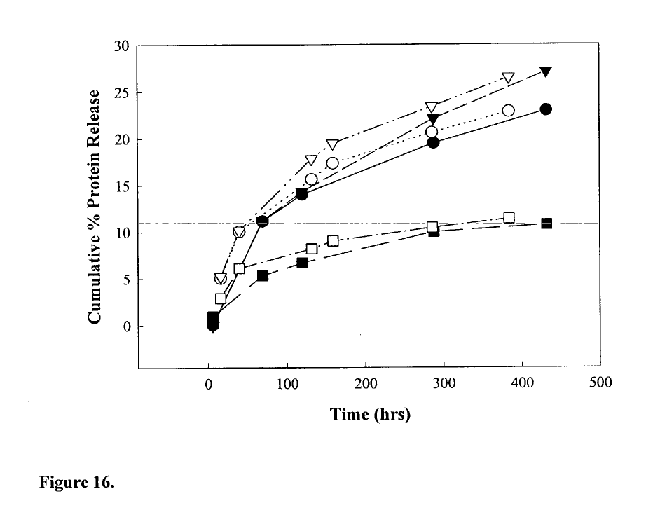

Effect of different wash buffers and release conditions on protein release from a single set of formulation conditions is shown in FIG. 16. Percent of the protein released from the pellet was determined under the following conditions: (1) HSA/IFN-α001 formulation washed with PBS and released in PBS Run 1 or Run 2; (2) HSA/IFN-α001 formulation washed with PBS and released in 3% Media Run 1 or Run 2; (3) HSA/IFN-α012 formulation washed with NaOAc and released in PBS Run 1; and HSA/ IFN-α001 formulation washed with NaOAc and released in PBS Run 2. Preparation of samples for run 1 and 2 was separated by 3 weeks.

For each run, 20 mg of HSA (Albutein lot # NG1829A) from a freshly prepared 50 mg/ml solution adjusted to pH 5.35 with acetic acid was placed in a 2.5 ml glass vial containing a micro stir bar. To this was added 40 μg of human IFN-α012 (1.36 mg/ml in 20 mM Tris pH 8.0, 200 mM NaCl, 6% glycerol) or 40 μg of human IFN-α001 (0.86 mg/ml in 20 mM Tris HCl, pH 8.0, 200 mM NaCl, 6% glycerol) and degassed water to give a final volume of 1.2 ml. N-Propanol (Fisher Sequencing Grade) was added to a final concentration of 40.1% w/v in two 600 μl steps with mixing. The glass vial containing the final formulation was covered with a rubber stopper and sealed with parafilm. The formulation was allowed to mature at 30(±2)° C. with moderate mixing for 18 hours. The formulation was then passed through a 22 G needle and split into two microcentrifuge tubes. After centrifugation at 13,000×g for 5 min, the supernate was removed and the pellet resuspended in either 1 ml PBS (PBS Wash) or 1 ml 25 mM NaOAc pH 5.35, 150 mM NaCl (NaOAc Wash). The pellets were allowed to sit in the wash solution from 10 to 15 min. The samples were then recentrifuged and the wash supernates were removed. To initiate release, the pellets were then resuspended in 1 ml of PBS with 0.02% Thimerosal. 10 μl was removed for analysis and the remainder was divided among 3 tubes. To these tubes were added either 10 μI PBS (PBS Release), 10 μl of five-day conditioned RPMI 1640 containing 10% FBS from the CTLL-2 cell line (3% Media Release). The final volume of each sample was approximately 330 μl, and the samples were incubated at 37° C. for release. Protein release was determined by BioRad Protein Assay in the microwell format at the indicated time points. Percent protein release was determined by solublizing each 10 μl pellet sample by addition of 40 μl 9M Urea, 50 mM NaCO₃, 50 mM NaCl pH 10.7, followed by determination of protein concentration by BioRad protein assay with correction vs. standards determined in diluted Urea solution.

The results from this experiment indicate that a low pH wash (NaOAc) slows release significantly when compared with a neutral wash (PBS). Furthermore, release into PBS appears similar to the release into PBS supplemented with 3% media (which had been conditioned by a T-cell line). Thus, the components in the RPMI 1640 base media, the fetal bovine serum or any substances secreted by the cell do not appear to alter the release in these conditions.

### EXAMPLE 16

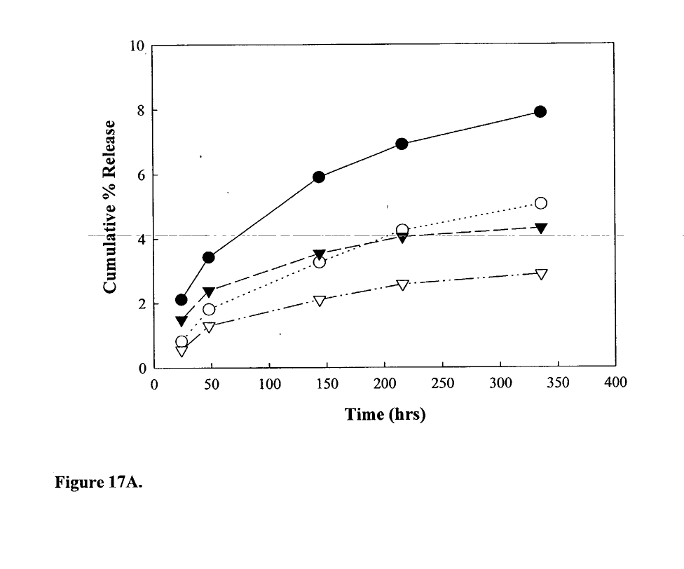

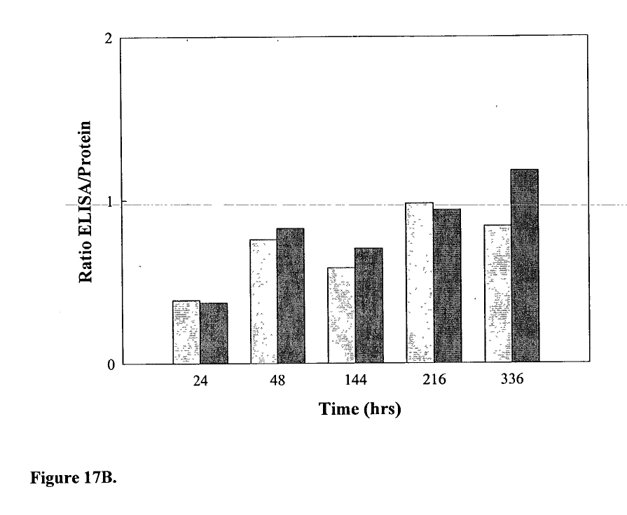

Release of total and immunoreactive protein from human IFN/HSA formulations is shown in FIG. 17A-B. Percent release of protein for human IFN-α001 and IFN-α012 was determined by BioRad Assay, while percent release of immunoreactive material for human IFN-α001 and IFN-α012 was determined by ELISA. In addition, the ratio of IFN immunoreactive material released to total protein released is calculated. Based on the results from Example 14, the formulations were chosen to contain 16% propanol, and the experiments were carried out under conditions that i have best of immunoreactive human IFN.

Formulations were prepared by mixing 300 μg of human IFN-α001 (0.96 mg/ml in 20 mM Tris HCl pH 8.0, 200 mM NaCl, 6% glycerol) or IFN-α012 (1.3 mg/ml in 20 mM Tris HCl, pH 8.0, 200 mM NaCl, 6% glycerol) with 25 μI (2 mg) of HSA (Albutein lot # NG1829A) from a 50 mg/ml stock pH adjusted to 5.35 with acetic acid, 25 μl of a 1 M NaOAc stock pH 5.35 and water to 1 ml. N-Propanol (Fisher Sequencing Grade) was added to a final concentration of 16% w/v (250 μl). After gentle mixing via the micropipet, the formulation was allowed to mature overnight at 30(±2)° C. for 18 hours without mixing. The precipitate was pelleted at 13,000×g for 10 minutes, resuspended in 1 ml of PBS to wash the pellet, and divided equally among 3 centrifuge tubes. After 10 minutes, the precipitate was recentrifuged and resuspended in 400 μl of PBS and incubated at 37° C. to initiate release. The total amount of protein in the pellet was determined by solublizing a 10 μl sample with 40 μl 9M Urea, 50 mM NaCO₃, 50 mM NaCl pH 10.7, and by BioRad protein assay with correction vs. standards determined in diluted Urea solution. The value for 100% activity is the amount of input INF activity less the activity remaining in the formulation supemate and wash supemate. Immunoreactive IFN was determined by ELISA (PBL Multispecies ELISA # 41105) using either human IFN-α001 or IFN-α012 as standards.

The results demonstrate controlled release of human IFNs from formulation particles. Immunoreactivity was used as a surrogate for biological activity. The ratio of human IFN immunoreactivity to total protein release suggests that most of the protein is active under these conditions. The lowest activity is observed in the first data point, perhaps due to preferential release of the small proportion of HSA that is incorporated into the particles.

### EXAMPLE 17

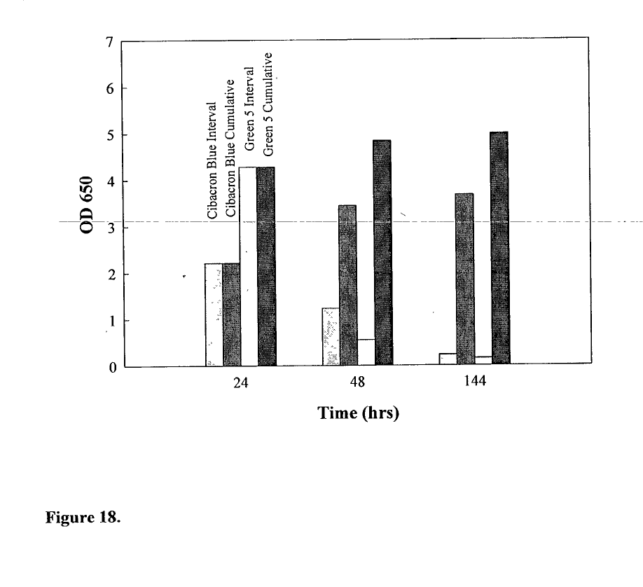

Comparison of release of small molecule dyes from HSA coformulations is shown in FIG. 18. Interval or Cumulative release of Cibacron Blue from HSA coformulations is compared with Interval or Cumulative release of Green 5 from HSA coformulations. These formulations were intended to demonstrate the utility of HSA for delivering co-formulated small molecules.

20 mg of HSA (Albutein lot # NG1829A) from a freshly prepared 50 mg/ml solution adjusted to pH 5.35 with acetic acid was placed in a 2.5 ml glass vial containing a micro stir bar. To this was added 150 μl of a 10 mg/ml solution of Cibacron Blue 3G-A (Sigma C 9534) in 25 mM NaOAc pH 5.35, or 150 μl of a 10 mg/ml solution of Green 5 (Sigma R 9253) in 25 mM NaOAc pH 5.35, and degassed water to give a final volume of 1.2 ml. N-Propanol (Fisher Sequencing Grade) was added to a final concentration of 40.1% w/v in two 600 μl steps with mixing. The final formulation (2.4 ml) was covered with a rubber stopper and sealed with parafilm. The formulation was allowed to mature at 30(±1)° C. with moderate mixing for 18 hours. Each formulation was then passed through a 22 G needle and split into two microcentrifuge tubes. After centrifugation at 13,000×g for 5 min, the supemate was removed and the pellet was resuspended in 1 ml PBS. The samples were then recentrifuged and the wash supernates were removed. To initiate release, the pellets were then resuspended in 1 ml of PBS with 0.02% Thimerosal. Release was determined by measuring the absorbance of the supernate at 650 nm.

The results from this experiment indicate that by using a blue and a green dye, release of the small molecules could be directly monitored by absorbance at 650 nm. These dyes serve as a model for bioactive small molecule pharmaceuticals. Under these formulation conditions, the dye alone does not form particles (data not shown).

### EXAMPLE 18

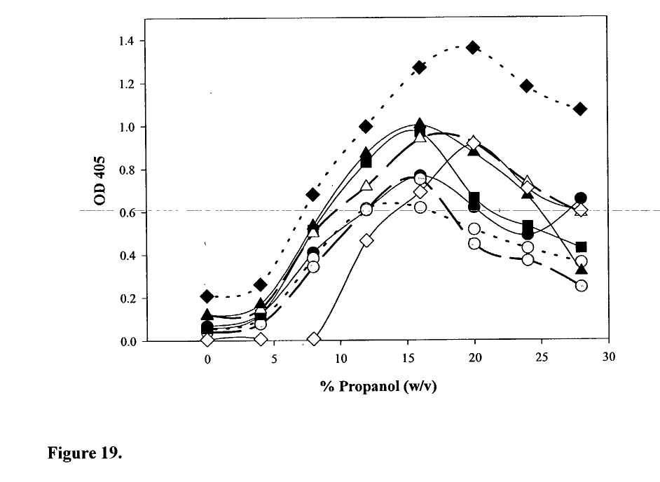

Formation of human IFN-α001 particles in the presence of stabilizers is shown in FIG. 19. Particle formation was monitored by measuring absorbance at 405 nm in the microplate assay. All samples contained 0.225 mg/ml human IFN-α001, 25 mM NaOAc pH 5.4, 25 mM NaCl, 0.2% glycerol final concentration. The final formulations were prepared by mixing 100 μl of a 2× concentrated stock of IFN in buffer and any stabilizers with 100 μl of a 2× concentrated propanol/water solution to initiate particle formation. The following final conditions were tested: (1) 25 mM NaCl; (2) 50 mM NaCl; (3) 100 mM NaCl; (4) 10 mM MgSO₄; (5) 0.5% Dextran 500,000; (6) 1.8% Trehalose; (7) 0.5% Tween 80; and (8) 0.05% SDS. These formulations were intended to demonstrate the effect of a variety of excipients, which are known to stabilize proteins, on the formation of human IFN particles.

Certain proteins, when formulated via a method disclosed herein, may benefit from the use of additional compounds to stabilize their structure and activity. Such stabilizers are compatible with the present methods. For example, as shown here, monovalent salt in final concentration of 25-100 mM, divalent salt exemplified by magnesium sulfate, sugars (dextran 500,000 and trehalose), and detergents exemplified by Tween 80 and SDS all allow particle formation to proceed.

### EXAMPLE 19

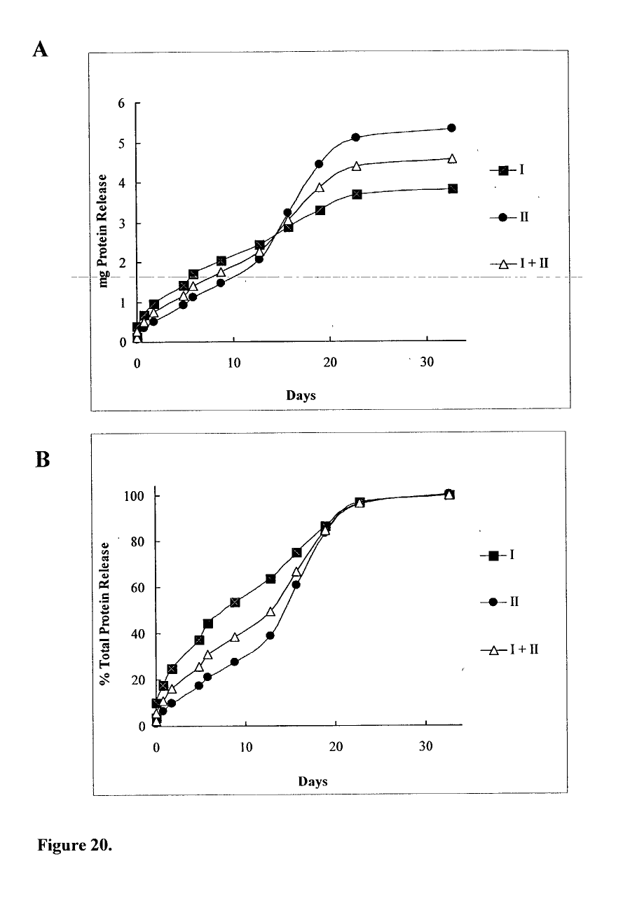

As disclosed herein, formulations prepared in different ways have different release kinetics. The combination of two or more formulations with different release kinetics may be used to generate a therapeutic composition having the desired overall release kinetics. Theoretical combination of two formulations to alter pattern of protein release is shown in FIG. 20. For Formulation I, Solution I consisted of 5 mg HSA (100 μl of 5% (w/v) HSA stock) and 47 μg of IFN-α001 (50 μl of 0.94 mg/ml stock). To this was added with stirring 59 μl H₂O, 90 μl of 0.01 M acetic acid, 16 μl 1M NaCl, and finally 237 μl t-butanol. The final formulation was stirred in a 2 ml glass vial for 22 hours at 24° C. prior to separating supernatant from precipitate. For Formulation II, Solution I consisted of 5 mg HSA (0.100 μl of 5% HSA stock) and 47 μg of IFN-α001 (50 μl of 0.94 mg/ml stock). To this was added with stirring 59 μl H₂O, 16 μl 1M NaCl, and finally 237 μl t-butanol. The final formulation was stirred in a 2 ml glass vial for 6 hours at 24° C. prior to separating supernatant from precipitate. The quantity of protein in washed precipitates was determined as described above. Release was performed in PBS/0.01% thimerosal. Theoretical release that would be obtained by mathematically averaging these two formulations is also presented.

The results indicate that formulations I and II each displayed fairly consistent rates of protein release to ˜23 days and completely dissolved by 33 days. However, the release pattern of Formulator II does reveal an increase in release rate beginning approximately at day 13. This type of pattern demonstrates that a series of formulations having early, medium, and late burst phases may be combined to yield a consistent rate of release. For example, when Formulation I is mathematically combined with Formulation II, the resulting release curve displays better consistency of release than either Formulation I or II alone.

### EXAMPLE 20

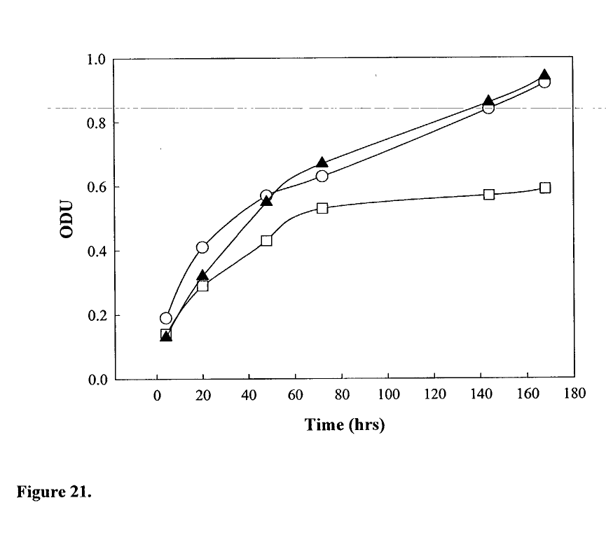

Release of a peptide and two small molecules from HSA co-formulations is shown in FIG. 21. Release of Cibacron Blue, Green 5, and a peptide Caspase I substrate from HSA co-formulations was monitored by absorbance of the Cibacron Blue and Green 5 at 650 nm and the peptide Caspase 1 substrate at 494 nm. 10 mg of HSA from a freshly prepared 50 mg/ml solution adjusted to pH 5.35 with acetic acid was placed in each of 4 microfuge tubes containing 25 μl of 1 M NaOAc pH 5.35. One tube then received 30 μl of a 10 mg/ml solution of Cibacron Blue 3G-A in 25 mM NaOAc pH 5.35, followed by 405 μl of degassed water and a total of 540 μl of n-propanol. The second tube received 30 μl of a 10 mg/ml solution of Green 5 in 25 mM NaOAc pH 5.35, followed by 405 μl of degassed water and a total of 540 μl of n-propanol. The third tube received 81 μl of 1.8 mM solution of the Caspase I substrate, FITC-Tyr-Val-Ala-Asp-Ala-Pro-Lys(DNP)-OH in 20 mM sodium carbonate, 354 μl of degassed water and a total of 540 μl of N-propanol. The fourth tube received 81 μl of 20 mM sodium carbonate, 354 μl of degassed water and 540 μl of N-propanol. In all cases, the propanol was added in two aliquots of 270 μl each followed by gentle mixing. The formulations were allowed to mature for 16 hours at 23° C. without mixing. Following maturation, the formulations were gently vortexed, passed several times through a 26 gauge needle, and centrifuged. The pelleted particles were washed with 1 ml of PBS and then 1 ml of 25 mM NaOAc pH 5.35, 150 mM NaCl by resuspension and centrifugation. Release was initiated by adding 1 ml of PBS containing 0.02% thimerosal and incubated at 37° C. Release of the two dyes was measured by absorbance at 650 nm and for the peptide by absorbance at 494 nm. The HSA only formulation was used as a control for absorbance at these wavelengths.

### EXAMPLE 21

Animal Study. This study demonstrates that formulations provide a sustained release in an animal model. Mice used for this study were Balb/c transgenic for the human Interferon α2b gene (Braun et al. 1997, Palleroni et al. 1997). Three mice were used for each formulation. As a, control, one set of mice was injected with 50 μg of soluble human IFN-α001. Based on published studies (Wang et al. 2001, Bohoslawec et al. 1986) and our own work, we expect soluble and/or released interferon α to have a half-life between 0.5 and 1.1 hours, and a volume of distribution of about 0.4 ml/g in the mouse. Thus, for a 20 g mouse, the volume of distribution should be ˜8 ml. At 24 hours after administration, given a half-life of 1 hr, we would predict that the serum concentration remaining from a 50 μg dose would be ˜1 pg/ml. In practice, all of the sera of this group of mice were below the detection limit of the ELISA of 40 pg/ml. To test our formulation strategy, we produced 5 different interferon formulations. Human IFN-α2b, human IFN-α001, human IFN-α012, and murine IFN-α formulations were allowed to mature for 18 hr. A fifth formulation contained human IFN-α001 and matured for 3 hours prior to centrifugation. The human IFN-α formulations were produced at 0.4 mg/ml protein with 25 mM NaOAc pH 5.4, 6 mM Tris pH 8.0, 50 mM NaCl, 1.25% glycerol, with particle formation intitiated by addition of N-propanol to 16%. The murine IFN-α formulation was produced at 0.4 mg/ml protein with 25 mM NaOAc pH 5.4, 4 mM HEPES pH 6.0, 50 mM NaCl, 1.25% glycerol, with particle formation initiated by addition of n-propanol to 16% final concentration (w/v). After particle formation, the mixture was centrifuged for 15 minutes at 3000×g and each pellet was resuspended in 1 ml of PBS. After incubation for 10 minutes at room temperature, the particles were spun down and washed again with PBS. Mice in groups of 3 each received 50 μl of formulation particles via subcutaneous (s.c.) injection on the back . Separate groups of 3 mice each received human IFN-α001 or human IFN-α012 18 hours formulations intramuscularly (i.m.) via 25 μl injection in the right rear leg muscle. Mice receiving human IFN-α001 or α012 each were dosed with ˜400 μg of interferon, while those receiving murine α or human α2b each were dosed with 200 μg of interferon. Serum was collected at 24 hours post injection, mice in all groups recieving a human interferon had immunoreactive interferon in their serum as determined by ELISA (PBL Multispecies ELISA # 41105). Average serum concentration of human interferon for the various groups of mice ranged from 350 to 1130 pg/ml. Mice injected with murine a particles gave no detectable levels of human interferon α in the serum. No interferon was detected at the 96 hours or 168 hours time points. These results indicate that the release formulations provide for a sustained level of interferon in animals at the early time point.

The values obtained in the mice are within the same order of magnitude of release rate observed for in vitro release from identical formulations.

1. A slow release formulation comprising one or more biologically active molecules from a solid composition prepared by exposure of the biologically active molecules to an organic solvent under conditions wherein a precipitate, lyophilate or crystal is formed.

2. A slow release formulation comprising precipitate, lyophilate or crystals of a polypeptide prepared by exposure of the polypeptide to an organic solvent, which polypeptide is released from the formulation in aqueous solution for a period of at least 7 days.

3. A formulation comprising precipitate, lyophilate or crystals of a biologically active polypeptide prepared by exposure of the polypeptide to a polar protic organic solvent, which formulation, when administered to a patient, releases said polypeptide at a rate providing an average steady state dosage of at least the ED₅₀ for the polypeptide for a period of at least 7 days.

4. The formulation of any of claims 1-3, wherein the organic solvent is an alcohol, an aldehyde, a ketone, a hydrocarbon, an aromatic hydrocarbon, or a mixture thereof.

5. The formulation of any of claims 1-3, wherein the organic solvent is an alcohol or mix of alcohols.

6. The formulation of claim 5, wherein the alcohol is a lower alcohol, or mixture thereof.

7. The formulation of claim 5, wherein the alcohol is selected from the group consisting of methanol, ethanol, isopropanol, n-propanol, n-butanol, isobutanol, and t-butanol, or a mixture thereof.

8. The formulation of any of claims 1-3, wherein the organic solvent is a polar protic solvent.

9. The formulation of any of claims 1-3, wherein the organic solvent is a water-miscible polar protic solvent.

10. The formulation of any of claims 1-3, wherein the biologically active molecules or polypeptides are released from the formulation in aqueous solution at a rate which provides an average steady state dosage of at least the ED₅₀ for the biologically active molecules or polypeptides for a period of at least 50 days.

11. The formulation of any of claims 1-3, wherein the organic solvent(s) are chosen such that, when administered to a patient, the solvent released from the formulation at a rate which remains at least one order of magnitude below the IC₅₀ for deleterious side effects, if any, of the solvent.

12. The formulation of claim 1, wherein biologically active molecule is a polymer selected from the group consisting of a protein, a peptide, a nucleic acid, an oligonucelotide, a carbohydrate, a ganglioside, or a glycan.

13. The formulation of any of claims 2-3, wherein the polypeptide is selected from the group consisting of cytokines, growth factors, somatotropin, growth hormones, colony stimulating factors, , erythropoietin, plasminogen activators, enzymes, T-cell receptors, surface membrane proteins, lipoproteins, clotting factors, anticlotting factors, tumor necrosis factors, transport proteins, homing receptors, and addressins.

14. The formulation of claim 13, wherein the polypeptide is selected from the group consisting of rennin; human growth hormone; bovine growth hormone; growth hormone releasing factor; parathyroid hormone; thyroid stimulating hormone; lipoproteins; α-1-antitrypsin; insulin; proinsulin; follicle stimulating hormone; calcitonin; luteinizing hormone; glucagon; a clotting factor such as factor VIIIC, factor IX, tissue factor, and von Willebrands factor; anti-clotting factors; atrial natriuretic factor; lung surfactant; a plasminogen activator; bombesin; thrombin; hemopoietic growth factor; tumor necrosis factor-α; tumor necrosis factor-β; enkephalinase; RANTES (regulated on activation normally T-cell expressed and secreted); human macrophage inflammatory protein (MIP-1-α); a serum albumin; mullerian-inhibiting substance; relaxin A-chain; relaxin β-chain; prorelaxin; gonadotropin-associated peptide; a microbial protein; DNase; inhibin; activin; vascular endothelial growth factor (VEGF); receptors for hormones or growth factors; integrin; protein A; protein D; rheumatoid factors; a neurotrophic factor; platelet-derived growth factor (PDGF); a fibroblast growth factor; epidermal growth factor (EGF); transforming growth factors (TGF); insulin-like growth factor-I; insulin-like growth factor-II; des(1-3)-IGF-I (brain IGF-I); insulin-like growth factor binding proteins; CD proteins; erythropoietin; osteoinductive factors; immunotoxins;; an interferon; colony stimulating factors (CSFs); interleukins (ILs); superoxide dismutase; T-cell receptors; surface membrane proteins; decay accelerating factor; antigens; transport proteins; homing receptors; addressing; regulatory proteins; immunoglobulin-like proteins; antibodies; and nucleases, or fragments thereof

15. The formulation of claim 1, wherein biologically active molecule is selected from the group consisting of a lipid and a sterol.

16. The formulation of claim 1, wherein biologically active molecule is a small organic compound.

17. The formulation of any of claims 1-3, which is a precipitate.

18. The formulation of any of claims 1-3, which is a lyophilate.

19. A formulation comprising a precipitate or lyophilate of a polypeptide, which precipitate or lyophilate includes at least 50 percent (molar) polar protic organic solvent(s), and which formulation, when administered to a patient, releases said polypeptide at a rate providing an average steady state dosage of at least the ED₅₀ for the polypeptide for a period of at least 7 days.

20. A medicament for administeration to an animal, comprising the formulation of any of claims 1-3.

21. The medicament of claim 20, for administeration to a mammal.

22. The medicament of claim 20, for administeration to a human.

23. A method for manufacturing a medicament comprising formulating the formulation of any of claims 1-3 with a pharmaceutically acceptable excipient.

24. A method method for manufacturing a slow release formulation of a biologically active molecule, comprising (a) exposing said biologically active molecules to an organic solvent, and (b) forming a precipitate, lyophilate or crystal.

25. A method for conducting a pharmaceutical business comprising:
(a) preparing a formulation of any of claims 1-3;
(b) providing marketing and/or product literature for instructing healthcare providers on the use of said formulations; and
(c) providing a distribution network for deliverying said formuation to healthcare providers.

26. A formulation comprising a first biopolymer and a biologically active molecule prepared by exposure of a mixture comprising the first biopolymer and the biologically active molecule to an organic solvent under conditions wherein a precipitate, lyophilate or crystal is formed.

27. The formulation of claim 26, wherein the biologically active molecule is selected from the group consisting of: a second biopolymer, a small organic compound and a small inorganic compound.

28. The formulation of claim 26, wherein the first biopolymer is selected from the group consisting of: a protein, a peptide, a nucleic acid, an oligonucelotide, a carbohydrate, a ganglioside, or a glycan.

29. The formulation of claim 26, wherein the first biopolymer has an insubstantial therapeutic effect.

30. The formulation of claim 26, wherein the first biopolymer has a substantial therapeutic effect.

31. A formulation of claim 26, wherein the first biopolymer and the biologically active molecule are released from the formulation in aqueous solution for a period of at least 7 days.

32. The formulation of any of claims 1-3 or 26, wherein the formulation further comprises a stabilizer.

33. The formulation of claim 1, wherein the exposure of the biologically active molecules to an organic solvent under conditions wherein a precipitate, lyophilate or crystal is formed comprises:
a) forming an aqueous mixture comprising the biologically active molecules; and
b) adding an amount of organic solvent sufficient to cause formation of a precipitate, lyophilate or crystal.

34. The formulation of claim 2, wherein the exposure of the polypeptide to an organic solvent comprises:
a) forming an aqueous mixture comprising the polypeptide; and
b) adding an amount of organic solvent sufficient to cause formation of a precipitate, lyophilate or crystal.

35. The formulation of claim 3, wherein the-exposure of the polypeptide to a polar protic organic solvent comprises:
a) forming an aqueous mixture comprising the polypeptide; and
b) adding an amount of polar protic organic solvent sufficient to cause formation of a precipitate, lyophilate or crystal.

36. The formulation of any of claims 33-35, wherein the aqueous mixture has a pH in the range of about 4 to about 9.

37. The formulation of any of claim 33-35, wherein the aqueous mixture comprises a salt at a concentration of about 5 mM to about 100 mM.

38. The formulation of claim 37, wherein the salt is a sodium salt.

39. The formulation of any of claims 33-35, wherein the aqueous mixture comprises an organic acid at a concentration of about 0.1 mM to about 10 mM.

40. The formulation of claim 39, wherein the organic acid is HOAc.

41. The formulation of any of claims 33-35, wherein the organic solvent is propanol.

42. The formulation of claim 41, wherein the amount of propanol added is sufficient to form a mixture having at least 8% propanol.

43. The method of claim 24, wherein exposing said biologically active molecules to an organic solvent comprises:
i) forming an aqueous mixture comprising the biologically active molecules; and
ii) adding an amount of organic solvent sufficient to cause formation of a precipitate, lyophilate or crystal.

44. The method of claim 24, wherein exposing said biologically active molecules to an organic solvent comprises:
i) forming a mixture comprising the biologically active molecules and the organic solvent; and
ii) adding an amount of an acid sufficient to cause formation of a precipitate, lyophilate or crystal.
